SEQUENCE LISTING (49 sequences)

SEQ ID NO: 1 - DNA, 20 bp, synthetic construct
  misc_feature 1..20: note = Synthetic Polynucleotide
  source 1..20: mol_type = other DNA
tccatgacgttcctgacgtt

SEQ ID NO: 2 - DNA, 21 bp, synthetic construct
  misc_feature 1..21: note = Synthetic Polynucleotide
  source 1..21: mol_type = other DNA
gctttcttgttggtgtaggtc

SEQ ID NO: 3 - DNA, 20 bp, synthetic construct
  misc_feature 1..20: note = Synthetic Polynucleotide
  source 1..20: mol_type = other DNA
tccatgacgttcctgatgct

SEQ ID NO: 4 - DNA, 21 bp, synthetic construct
  misc_feature 1..21: note = Synthetic Polynucleotide
  source 1..21: mol_type = other DNA
gctttcttgttggtgtaggtc

SEQ ID NO: 5 - DNA, 20 bp, synthetic construct
  misc_feature 1..20: note = Synthetic Polynucleotide
  source 1..20: mol_type = other DNA
tccatgacgttcctgatgct

SEQ ID NO: 6 - DNA, 20 bp, synthetic construct
  misc_feature 1..20: note = Synthetic Polynucleotide
  source 1..20: mol_type = other DNA
tccatgacgttcctgatgct

SEQ ID NO: 7 - DNA, 20 bp, synthetic construct
  misc_feature 1..20: note = Synthetic Polynucleotide
  source 1..20: mol_type = other DNA
tccatgacgttcctgatgct

SEQ ID NO: 8 - DNA, 20 bp, synthetic construct
  misc_feature 1..20: note = Synthetic Polynucleotide
  source 1..20: mol_type = other DNA
tccatgacgttcctgatgct

SEQ ID NO: 9 - DNA, 20 bp, synthetic construct
  misc_feature 1..20: note = Synthetic Polynucleotide
  source 1..20: mol_type = other DNA
tccatgacgttcctgacgtt

SEQ ID NO: 10 - DNA, 20 bp, synthetic construct
  misc_feature 1..20: note = Synthetic Polynucleotide
  source 1..20: mol_type = other DNA
tccatgacgttcctgacgtt

SEQ ID NO: 11 - DNA, 20 bp, synthetic construct
  misc_feature 1..20: note = Synthetic Polynucleotide
  source 1..20: mol_type = other DNA
tccatgacgttcctgacgtt

SEQ ID NO: 12 - DNA, 20 bp, synthetic construct
  misc_feature 1..20: note = Synthetic Polynucleotide
  source 1..20: mol_type = other DNA
tccatgacgttcctgacgtt

SEQ ID NO: 13 - RNA, 18 nt, synthetic construct
  misc_feature 1..18: note = Synthetic Polynucleotide
  source 1..18: mol_type = other RNA
ccgtctgttgtgtgactc

SEQ ID NO: 14 - RNA, 20 nt, synthetic construct
  misc_feature 1..20: note = Synthetic Polynucleotide
  source 1..20: mol_type = other RNA
gccaccgagccgaaggcacc

SEQ ID NO: 15 - RNA, 20 nt, synthetic construct
  misc_feature 1..20: note = Synthetic Polynucleotide
  source 1..20: mol_type = other RNA
tatatatatatatatatata

SEQ ID NO: 16 - RNA, 20 nt, synthetic construct
  misc_feature 1..20: note = Synthetic Polynucleotide
  source 1..20: mol_type = other RNA
ttattattattattattatt

SEQ ID NO: 17 - RNA, 20 nt, synthetic construct
  misc_feature 1..20: note = Synthetic Polynucleotide
  source 1..20: mol_type = other RNA
ttttattttattttatttta

SEQ ID NO: 18 - RNA, 20 nt, synthetic construct
  misc_feature 1..20: note = Synthetic Polynucleotide
  source 1..20: mol_type = other RNA
tgtgtgtgtgtgtgtgtgtg

SEQ ID NO: 19 - RNA, 20 nt, synthetic construct
  misc_feature 1..20: note = Synthetic Polynucleotide
  source 1..20: mol_type = other RNA
ttgttgttgttgttgttgtt

SEQ ID NO: 20 - RNA, 20 nt, synthetic construct
  misc_feature 1..20: note = Synthetic Polynucleotide
  source 1..20: mol_type = other RNA
tttgtttgtttgtttgtttg

SEQ ID NO: 21 - RNA, 24 nt, synthetic construct
  misc_feature 1..24: note = Synthetic Polynucleotide
  source 1..24: mol_type = other RNA
ttatttatttatttatttatttat

SEQ ID NO: 22 - RNA, 24 nt, synthetic construct
  misc_feature 1..24: note = Synthetic Polynucleotide
  source 1..24: mol_type = other RNA
ttgtttgtttgtttgtttgtttgt

SEQ ID NO: 23 - RNA, 20 nt, synthetic construct
  misc_feature 1..20: note = Synthetic Polynucleotide
  source 1..20: mol_type = other RNA
gcccgtctgttgtgtgactc

SEQ ID NO: 24 - RNA, 18 nt, synthetic construct
  misc_feature 1..18: note = Synthetic Polynucleotide
  source 1..18: mol_type = other RNA
gtccttcaagtccttcaa

SEQ ID NO: 25 - DNA, 20 bp, synthetic construct
  misc_feature 1..20: note = Synthetic Polynucleotide
  source 1..20: mol_type = other DNA
ggtgcatcgatgcagggggg

SEQ ID NO: 26 - DNA, 22 bp, synthetic construct
  misc_feature 1..22: note = Synthetic Polynucleotide
  source 1..22: mol_type = other DNA
tccatggacgttcctgagcgtt

SEQ ID NO: 27 - DNA, 22 bp, synthetic construct
  misc_feature 1..22: note = Synthetic Polynucleotide
  source 1..22: mol_type = other DNA
tcgtcgttcgaacgacgttgat

SEQ ID NO: 28 - DNA, 22 bp, synthetic construct
  misc_feature 1..22: note = Synthetic Polynucleotide
  source 1..22: mol_type = other DNA
tcgtcgacgatccgcgcgcgcg

SEQ ID NO: 29 - DNA, 20 bp, synthetic construct
  misc_feature 1..20: note = Synthetic Polynucleotide
  source 1..20: mol_type = other DNA
ggggtcaacgttgagggggg

SEQ ID NO: 30 - DNA, 24 bp, synthetic construct
  misc_feature 1..24: note = Synthetic Polynucleotide
  source 1..24: mol_type = other DNA
tcgtcgttttgtcgttttgtcgtt

SEQ ID NO: 31 - DNA, 22 bp, synthetic construct
  misc_feature 1..22: note = Synthetic Polynucleotide
  source 1..22: mol_type = other DNA
tcgtcgttgtcgttttgtcgtt

SEQ ID NO: 32 - DNA, 20 bp, synthetic construct
  misc_feature 1..20: note = Synthetic Polynucleotide
  source 1..20: mol_type = other DNA
gggggacgatcgtcgggggg

SEQ ID NO: 33 - DNA, 21 bp, synthetic construct
  misc_feature 1..21: note = Synthetic Polynucleotide
  source 1..21: mol_type = other DNA
ggggacgacgtcgtggggggg

SEQ ID NO: 34 - DNA, 22 bp, synthetic construct
  misc_feature 1..22: note = Synthetic Polynucleotide
  source 1..22: mol_type = other DNA
tcgtcgttttcggcgcgcgccg

SEQ ID NO: 35 - DNA, 25 bp, synthetic construct
  misc_feature 1..25: note = Synthetic Polynucleotide
  source 1..25: mol_type = other DNA
tcgtcgtcgttcgaacgacgttgat

SEQ ID NO: 36 - DNA, 20 bp, synthetic construct
  misc_feature 1..20: note = Synthetic Polynucleotide
  source 1..20: mol_type = other DNA
tccatgacgttcctgatgct

SEQ ID NO: 37 - DNA, 20 bp, synthetic construct
  misc_feature 1..20: note = Synthetic Polynucleotide
  source 1..20: mol_type = other DNA
tccatgacgttcctgacgtt

SEQ ID NO: 38 - DNA, 24 bp, synthetic construct
  misc_feature 1..24: note = Synthetic Polynucleotide
  source 1..24: mol_type = other DNA
tcgtcgttttgtcgttttgtcgtt

SEQ ID NO: 39 - DNA, 20 bp, synthetic construct
  misc_feature 1..20: note = Synthetic Polynucleotide
  source 1..20: mol_type = other DNA
tccatgacgttcctgatgct

SEQ ID NO: 40 - DNA, 20 bp, synthetic construct
  misc_feature 1..20: note = Synthetic Polynucleotide
  source 1..20: mol_type = other DNA
tccatgacgttcctgacgtt

SEQ ID NO: 41 - DNA, 24 bp, synthetic construct
  misc_feature 1..24: note = Synthetic Polynucleotide
  source 1..24: mol_type = other DNA
tcgtcgttttgtcgttttgtcgtt

SEQ ID NO: 42 - DNA, 20 bp, synthetic construct
  misc_feature 1..20: note = Synthetic Polynucleotide
  source 1..20: mol_type = other DNA
tccatgacgttcctgacgtt

SEQ ID NO: 43 - DNA, 20 bp, synthetic construct
  misc_feature 1..20: note = Synthetic Polynucleotide
  source 1..20: mol_type = other DNA
tccatgacgttcctgacgtt

SEQ ID NO: 44 - DNA, 20 bp, synthetic construct
  misc_feature 1..20: note = Synthetic Polynucleotide
  source 1..20: mol_type = other DNA
tccatgacgttcctgatgct

SEQ ID NO: 45 - DNA, 20 bp, synthetic construct
  misc_feature 1..20: note = Synthetic Polynucleotide
  source 1..20: mol_type = other DNA
tccatgacgttcctgatgct

SEQ ID NO: 46 - DNA, 24 bp, synthetic construct
  misc_feature 1..24: note = Synthetic Polynucleotide
  source 1..24: mol_type = other DNA
tcgtcgttttgtcgttttgtcgtt

SEQ ID NO: 47 - DNA, 24 bp, synthetic construct
  misc_feature 1..24: note = Synthetic Polynucleotide
  source 1..24: mol_type = other DNA
tcgtcgttttgtcgttttgtcgtt

SEQ ID NO: 48 - DNA, 21 bp, synthetic construct
  misc_feature 1..21: note = Synthetic Polynucleotide
  source 1..21: mol_type = other DNA
gctttcttgttggtgtaggtc

SEQ ID NO: 49 - DNA, 21 bp, synthetic construct
  misc_feature 1..21: note = Synthetic Polynucleotide
  source 1..21: mol_type = other DNA
gctttcttgttggtgtaggtc

(19) United States
(12) Patent Application Publication
(10) Pub. No.: US 2025/0283096 A1
(43) Pub. Date: Sep. 11, 2025

(54) SPHERICAL NUCLEIC ACID-BASED CONSTRUCTS AS IMMUNOSTIMULATORY AGENTS FOR PROPHYLACTIC AND THERAPEUTIC USE

(71) Applicant: Flashpoint Therapeutics, Inc., Palo Alto, CA (US)

(72) Inventors: Aleksandar Filip Radovic-Moreno, Evanston, IL (US); Christopher C. Mader, Cambridge, MA (US); Subbarao Nallagatla, Chicago, IL (US); Warefta Hasan, Houston, TX (US); Aaron Love, Chicago, IL (US); Sergei Gryaznov, San Mateo, CA (US)

(73) Assignee: Flashpoint Therapeutics, Inc., Palo Alto, CA (US)

(21) Appl. No.: 19/217,201

(22) Filed: May 23, 2025

Related U.S. Application Data
(63) Continuation of application No. 17/066,134, filed on Oct. 8, 2020, now abandoned.
Application No. 17/066,134 is a continuation of application No. 15/980,428, filed on May 15, 2018, now Pat. No. 10,837,018.
Application No. 15/980,428 is a continuation of application No. 14/907,430, filed on Jan. 25, 2016, now Pat. No. 10,894,963.
(60) Provisional application No. 61/858,558, filed on Jul. 25, 2013.

(51) Int. Cl. C12N 15/117; A61K 39/00; A61K 39/39; B82Y 5/00

(52) U.S. Cl. CPC C12N 15/117; A61K 39/39; A61K 2039/55561; B82Y 5/00; C12N 2310/17; C12N 2320/30; C12N 2320/32

(57) ABSTRACT

Aspects of the invention relate to spherical nucleic acid-based constructs and related methods and compositions thereof. The compositions of the invention are useful for activating agonists of nucleic acid interacting complexes, such as TLRs, stimulating an immune response, and treating diseases such as infectious disease, cancer, allergies, allergic diseases, and autoimmune disease

### RELATED APPLICATIONS

This application is a continuation of U.S. application Ser. No. 14/907,430, filed Jan. 25, 2016, entitled “SPHERICAL NUCLEIC ACID-BASED CONSTRUCTS AS IMMUNOSTIMULATORY AGENTS FOR PROPHYLACTIC AND THERAPEUTIC USE”, which is a national stage filing under 35 U.S.C. 371 of International Patent Application Serial No. PCT/US2014/048291, filed Jul. 25, 2014, entitled “SPHERICAL NUCLEIC ACID-BASED CONSTRUCTS AS IMMUNOSTIMULATORY AGENTS FOR PROPHYLACTIC AND THERAPEUTIC USE”, which is a Non-Prov of Prov (35 USC 119(e)) of U.S. Application Ser. No. 61/858,558, filed Jul. 25, 2013, entitled “SPHERICAL NUCLEIC ACID-BASED CONSTRUCTS AS IMMUNOSTIMULATORY AGENTS FOR PROPHYLACTIC AND THERAPEUTIC USE”. The entire contents of these applications are incorporated herein by reference in their entirety.

### FIELD OF INVENTION

The invention relates to nanoscale constructs of agonists of nucleic acid-interacting complexes, such as agonists of TLR, as well as methods and compositions thereof.

### BACKGROUND OF INVENTION

The immune system is a highly evolved, exquisitely precise endogenous mechanism for clearing foreign, harmful, and unnecessary material including pathogens and senescent or malignant host cells. It is known that modulating the immune system for therapeutic or prophylactic purposes is possible by introducing compounds that modulate the activity of specific immune cells. A primary example is vaccines, which have shown the ability to induce protection against pathogens as well as cancerous cells. The first modern vaccine formulations included live/attenuated or inactivated pathogens, but these were deemed too toxic in many instances or did not provide protective immunity. Purified protein derivatives and other antigenic subunit vaccine strategies have been pursued, but these typically lead to mildly protective or inefficient responses. It is now appreciated that effective immunity, in most instances, is known to require use of immunostimulatory compounds, which, among other things, provide the necessary signals to induce more robust, specific, and long-lived responses, including cell-mediated immunity and immunologic memory. The nature of these responses can be modulated by the type of immunostimulatory compound(s) introduced. Indeed, it has been postulated that immunostimulatory compounds administered together in the presence of appropriate antigenic stimuli can be used to elicit a wide variety of immune responses, with the potential to treat or prevent various ailments, including infectious diseases and cancer. These can also potentially be used to vaccinate immunocompromised populations, such as children and the elderly.¹

Existing vaccines fail to induce effective immune responses in a variety of diseases with critical worldwide impact, including AIDS, malaria, Chlamydia, various malignancies and allergies or allergic diseases, such as asthma. Among the immunostimulatory compounds being developed, agonists of Toll-like receptors (TLR) have demonstrated outstanding potential. Agonists of TLR4, such as monophosphoryl lipid A (MPL) have reached late stages of clinical trials and approval in various countries in some instances.² Despite these promising results, there is still a clear and significant need for compounds which can safely and effective induce responses that can clear intracellular pathogens and cancers, such as inducers of cell-mediated immunity. Agonists of TLR 3, TLR 7/8 and TLR 9 have excellent potential due to their potent ability to induce Th1 cell-mediated immune responses. A synthetic TLR 7/8 agonist, imiquimod, has been approved to treat various skin diseases, including superficial carcinomas and genital warts, and is being developed for a variety of other indications. Similarly, agonists of TLR 9 are in various stages of clinical development, for treatment of various diseases with large unmet medical needs. However, concerns due to lack of efficacy, off-target phosphorothioate effects, and toxicity have slowed effective clinical translation of TLR 7/8 and 9 agonists.

### SUMMARY OF INVENTION

Described herein are novel methods and compositions for enhancing immune responses and activating nucleic acid interacting complexes such as TLRs using a nanoscale construct. Aspects of the invention relate to nanoscale constructs having a corona of an agonist of nucleic acid-interacting complexes wherein the surface density of the agonist of nucleic acid-interacting complexes is at least 0.3 pmol/cm².

In other aspects the invention is a nanoscale construct having a corona of an agonist of nucleic acid-interacting complexes, and an antigen incorporated into the corona. In some embodiments the surface density of the antigen is at least 0.3 pmol/cm². In other embodiments the antigen includes at least two different types of antigen.

In yet other aspects the invention is a nanoscale construct having a corona with at least two agonists of nucleic acid-interacting complexes incorporated, wherein the agonists are selected from the group consisting of TLR 3, 7/8, and/or 9 agonists.

In some embodiments the agonist of nucleic acid-interacting complexes contains a spacer.

In other embodiments the agonist of nucleic acid-interacting complexes is RNA or DNA. The agonists of nucleic acid-interacting complexes may be, for instance, a double stranded RNA, such as poly(I:C). Alternatively the agonist of nucleic acid-interacting complexes may be a single stranded RNA such as an RNA containing UUG-motifs. In some embodiments the agonist of nucleic acid-interacting complexes is an unmethylated deoxyribonucleic acid, such as a CpG oligonucleotide.

The nanoscale construct, in some embodiments, contains a nanoparticle core at the center of the corona which is optionally metallic. The metallic core may be selected from the group consisting of gold, silver, platinum, aluminum, palladium, copper, cobalt, indium, nickel and mixtures thereof. In some embodiments the nanoparticle core comprises gold. In other embodiments the nanoscale construct is degradable.

In certain embodiments, the diameter of the nanoscale construct is from 1 nm to about 250 nm in mean diameter, about 1 ran to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, or about 1 nm to about 10 nm in mean diameter.

In other aspects the invention is a nanoscale construct of a corona of an agonist of nucleic acid-interacting complexes, wherein the agonist is nucleic acid having at least one phosphodiester internucleotide linkage. In some embodiments the agonist is a CpG oligonucleotide. In other embodiments each internucleotide linkage of the nucleic acid is a phosphodiester linkage.

In embodiments of the invention the corona is a spherical corona.

A vaccine composed of a nanoscale construct described herein and a carrier is provided according to other aspects of the invention.

A method for delivering a therapeutic agent to a cell by delivering the nanoscale construct of the invention to the cell is provided in other aspects.

A method for regulating expression of a target molecule is provided in other aspects of the invention. The method involves delivering the nanoscale construct of the invention to the cell. In some embodiments the target molecule is a TLR selected from the group consisting of TLR3, 7, 8, and 9.

A method for activating a TLR by delivering the nanoscale construct as described herein to the cell is provided in other aspects of the invention.

According to other aspects the invention is a method of treating a subject, involving administering to the subject the nanoscale construct as described herein in an effective amount to stimulate an immune response. In some embodiments the subject has an infectious disease, a cancer, an autoimmune disease, allergy, or an allergic disease such as asthma.

In yet other embodiments, the invention is a method of inducing an immune response in a subject, by administering to the subject a nanoscale construct of a corona of an agonist of nucleic acid-interacting complexes, wherein the agonist is nucleic acid having at least one phosphodiester internucleotide linkage in an effective amount to stimulate an immune response.

In certain embodiments, the method involves delivering a therapeutic or detection modality to a cell.

Further aspects of the invention relate to a kit comprising: a nanoscale construct optionally including a nanoparticle core; and having an agonist of nucleic acid-interacting complexes and instructions for assembly of an agonist of nucleic acid-interacting complexes-corona. In certain embodiments, the kit further comprises instructions for use.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1B show a schematic non-limiting example of a nanoscale construct of the invention. FIG. 1A. A general structure of an adjuvant nanoscale construct having a core and one or more agonists of nucleic acid interacting complexes, such as TLR agonists bound thereto is shown. FIG. 1B. A general structure of an adjuvant nanoscale construct having a core and one or more TLR agonists and one or more antigens bound thereto is shown.

FIG. 2 is a set of graphs depicting markedly enhanced potency in macrophages of the nanoscale constructs of the invention over agonists of nucleic acid-interacting complexes (CpG oligonucleotides) in solution.

FIG. 3 is a set of graphs depicting markedly enhanced potency in macrophages of the nanoscale constructs of the invention over agonists of nucleic acid-interacting complexes (CpG oligonucleotides) in solution following overnight incubation.

FIG. 4 is a set of graphs depicting an enhanced level of cytokine secretion with the nanoscale constructs of the invention over agonists of nucleic acid-interacting complexes (CpG oligonucleotides) in solution. The effect on cytokine induction was examined for both oligonucleotides having phosphodiester and phosphorothioate internucleotide linkages in both the nanoscale construct and the TRL agonist groups.

FIG. 5 is a line and bar graph depicting TLR9 activation in response to stimulation with a nanoscale construct of the invention having a phosphodiester CpG oligonucleotide in comparison with phosphodiester and phosphorothioate CpG oligonucleotides in solution.

FIG. 6 is a set of graphs depicting multiple fold increase in potency of a nanoscale construct of the invention over several different CpG oligonucleotide sequences. CpG 1826 is SEQ ID NO: 1 and the rplV below it is SEQ ID NO: 2. CpG 1668 is SEQ ID NO: 3 and the rplV below it is SEQ ID NO: 4.

FIGS. 7A-7B are a set of graphs depicting the effects of modulating nanoscale construct core size suggests on the enhancement of agonist activity.

FIG. 8 shows a graph depicting more rapid and sustained activation than CpG oligonucleotide.

FIG. 9 is a set of graphs depicting the ability of phosphorothioate modifications to modulate agonist activity in a sequence-dependent manner. For CpG 1668: PO is SEQ ID NO: 5, C*G is SEQ ID NO: 6, 5PS2/C*G is SEQ ID NO: 7, and PS is SEQ ID NO: 8. For CpG 1826: PO is SEQ ID NO: 9, C*G is SEQ ID NO: 10, 5PS2/C*G is SEQ ID NO: 11, and PS is SEQ ID NO: 12.

FIG. 10 is a set of graphs depicting the ability of oligonucleotide loading density to affect agonist activity.

FIG. 11 shows a graph depicting a time course of activation of CpG PO/PO nanoscale constructs. The tested constructs are not activated until >4 hr of incubation.

FIG. 12 shows a graph demonstrating that 5′Chol CpG PO nanoscale constructs show activation in low nM range, and 5′C18 abrogated the activity.

FIG. 13 is a set of graphs demonstrating that pre-plated macrophages are more primed for subsequent activation.

FIG. 14 is a set of graphs demonstrating low levels of IFN-gamma secretion by macrophages.

FIG. 15 shows a representation of an immunotherapeutic SNA (AST-008). FIG. 15 shows that the SNAs can co-present a therapeutic vaccine antigen and adjuvant on a single nanoparticle, and may simultaneously target multiple immunostimulatory receptors (e.g. TLR 3, 4, 7/8, 9).

FIG. 16 is a schematic demonstrating how AST-008 can enter endosomes via triggered endocytosis, where it then can be used for versatile immune system stimulation. Within the endosome, AST-008 stimulates immune system signaling via the TLR 9 receptor, a molecular target for SNA therapy, leading to both innate and adaptive immune responses. AST-008 may also target TLR 3, 4, 7/8, resulting in innate and adaptive immune responses.

FIGS. 17A-17B are a set of graphs showing that AST-008 induces higher pro-inflammatory responses than corresponding CpG oligodeoxynucleotides (oligo) in vitro. FIG. 17A shows the expression levels of TNF, IL-12, and IL-6 induced by CTL oligo, CTL SNA, CpG 1826, and AST-008. FIG. 17B presents the NF-κB activation stemming from the indicated agents.

FIG. 18 demonstrates that AST-008 targets draining lymph nodes after administration of a single subcutaneous dose. AST-008 was silver-stained to enhance light scattering of the gold core, and then counterstained with eosin. 4× bright field magnification was used.

FIG. 19 is a graph illustrating the in vivo activity of AST-008. Mice were given a 50 μL bolus tail vein (intravenous) injection of 5.1 nmol solution (AST-008-po, AST-008-ps, CpG 1826-po, CpG 1826-ps, GpC-po SNA, GpC-ps SNA, GpC-po, or GpC-ps) and then analyzed for IL-12 expression 1, 3, and 6 hours after injection (24 mice per group, 3 per each time point). IL-12 levels are expressed as the fold over PBS. AST-008 architecture enhances the induction of IL-12 by approximately 20-fold over free oligodeoxynucleotides, and the effect was sustained for over six hours after the initial administration.

FIGS. 20A-20C consist of a pair of graphs and a chart that demonstrate that AST-008 induces both a balanced Th1/Th2 response (FIG. 20A) and a higher IgG2a antibody (FIG. 20B) response than alum or CpG oligonucleotides. The results are tabulated in FIG. 20C. **p<0.01.

FIGS. 21A-21B show that AST-008 induces cellular responses more effectively than alum or CpG oligonucleotides. FIG. 21A schematically represents the protocol: splenocytes were grown for 28 days, challenged on Day 0 and Day 21, and then restimulated with SIINFEKL and probed for INF-γ with ELISPOT on Day 28. FIG. 21B is a graph depicting the results. ****p<0.0001.

FIGS. 22A-22B demonstrate that AST-008 induces a profound tumor-clearing immune response in an in vivo lymphoma model. FIG. 22A illustrates the protocol: the right flanks of C57BL/6 mice were injected with 1×10⁶ E.G7-OVA lymphoma (11 per group). The mice were then challenged three times with 100 μg OVA s.c., 1.8 μg OVA₂₅₇₋₂₆₄ s.c., and 0.92 nmol oligo in AST-008, and sacrificed at 2000 mm³. FIG. 22B is a graph of the results. *p<0.05 using Two-way ANOVA.

FIGS. 23A-23B show that AST-008 exhibits superior anti-tumor activity and longer survival than CpG oligodeoxynucleotides. The graphs show the tumor volume (FIG. 23A) and percent survival (FIG. 23B) after C57BL/6 mice were injected with 1×10⁶ E.G7-OVA lymphoma in their right flanks (11 per group) and then were challenged three times with PBS, PBS and OVA, CpG 1826 and OVA, or AST-008 and OVA. *p<0.05.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The present invention, in some aspects, overcomes several major hurdles encountered by conventional TLR 3, TLR 7/8, and TLR 9 agonists by achieving faster activation, creating a multivalent structure, changing cellular distribution, and facilitating simple and scalable synthesis of various adjuvant and antigen-containing structures, among others. The constructs of the invention result in more effective vaccines for prophylactic or therapeutic uses in treating a wide variety of diseases/Infections including, for example, AIDS, malaria, Chlamydia, Campylobacter, cytomegalovirus, dengue, Epstein-Barr mononucleosis, foot and mouth disease, rabies, Helicobacter pylori gastric ulcers, hepatitis A, B, C, herpes simplex, influenza, leishmaniasis, cholera, diphtheria, Haemophilus influenza, meningococcal meningitis, plague, pneumococcal pneumonia, tetanus, typhoid fever, respiratory syncitial virus, rhinovirus, schistosomiasis, Shigella, Streptococcus group A and B, tuberculosis, Vibrio cholera, Salmonella, aspergillus, blastomyces, Histoplasma, Candida, Cryptococcus, Pneumocystis, and urinary tract infections; various food allergies such as peanut, fruit, garlic, oats, meat, milk, fish, shellfish, soy, tree nut, wheat, gluten, egg, sulphites; various drug allergies such as to tetracycline, Dilantin, carbamazepine, penicillins, cephalosporins, sulfonamides, NSAIDs, intravenous contrast dye, local anesthetics; autoimmune diseases such as multiple sclerosis, lupus, inflammatory bowel disease, Crohn's disease, ulcerative colitis, asthma, and COPD; and cancers such as melanoma, breast cancer, prostate cancer, bladder cancer, NSCLC, glioblastoma multiforme, among others.

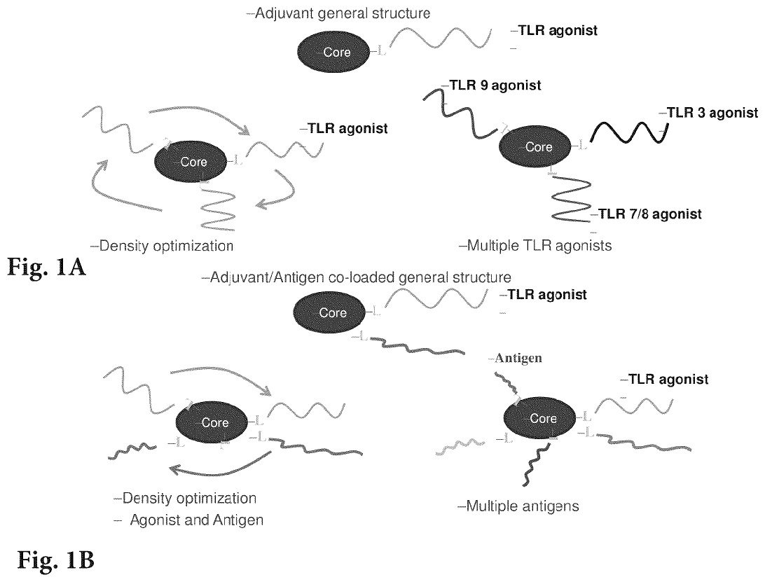

A set of exemplary nanoscale constructs of the invention is shown in the schematic of FIGS. 1A-1B. The platform described herein is useful for loading one or multiple agonists of nucleic acid-interacting complexes (A) or one or multiple agonists of nucleic acid-interacting complexes and antigen (B). Optimization of: (1) nucleic acid interacting complex, such as a TLR that is targeted (TLR 3, 7/8, and/or 9), (2) density of agonist of nucleic acid-interacting complexes, (3) antigen density, (4) multiple antigen presentation, (5) core composition, size, and charge, and (6) core linker chemistry “L”, and (7) agonist chemical structure is expected to yield novel paradigms in vaccine development. In particular, agonists of nucleic acid-interacting complexes include double stranded RNA (such as poly(I:C), TLR 3), single stranded RNA (such as strands containing UUG-motifs, TLR 7/8), and unmethylated deoxyribonucleic acid and derivatives (such as strands containing CpG motifs).

Aspects of the invention relate to nanoscale constructs. A nanoscale construct refers to a nanometer sized construct having one or more nucleic acids held in a geometrical position. The nanoscale construct typically is referred to as a corona of a set of nucleic acids. A corona, as used herein, refers to an exterior shell composed of nucleic acid molecules. The corona may have a nanoparticle core composed of nucleic acids or other materials, such as metals. Alternatively, the corona may simply be a set of nucleic acids arranged in a geometric shape with a hollow core, i.e. a 3-dimensionally shaped layer of nucleic acids. Typically, but not always, the corona has a spherical shape.

In the instance, when the corona includes a nanoparticle core the nucleic acids may be linked directly to the core. Some or all of the nucleic acids may be linked to other nucleic acids either directly or indirectly through a covalent or non-covalent linkage. The linkage of one nucleic acid to another nucleic acid may be in addition to or alternatively to the linkage of that nucleic acid to a core. One or more of the nucleic acids may also be linked to other molecules such as an antigen.

When the corona does not include a nanoparticle core, the nucleic acids may be linked to one another either directly or indirectly through a covalent or non-covalent linkage. In some embodiments the corona that does not include a nanoparticle core may be formed by layering the nucleic acids on a lattice or other dissolvable structure and then dissolving the lattice or other structure to produce an empty center.

As used herein, the nanoscale construct is a construct having an average diameter on the order of nanometers (i.e., between about 1 nm and about 1 micrometer. For example, in some instances, the diameter of the nanoparticle is from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, about 1 nm to about 20 nm in mean diameter, about 1 nm to about 10 nm in mean diameter, about 5 nm to about 150 nm in mean diameter, about 5 to about 50 nm in mean diameter, about 10 to about 30 nm in mean diameter, about 10 to 150 nm in mean diameter, about 10 to about 100 nm in mean diameter, about 10 to about 50 nm in mean diameter, about 30 to about 100 nm in mean diameter, or about 40 to about 80 nm in mean diameter.

In some instances the corona includes a nanoparticle core that is attached to one or more agonists of nucleic acid-interacting complexes and/or antigens. As used herein, a nanoparticle core refers to the nanoparticle component of a nanoparticle construct, without any attached modalities. In some instances, the nanoparticle core is metallic. It should be appreciated that the nanoparticle core can comprise any metal. Several non-limiting examples of metals include gold, silver, platinum, aluminum, palladium, copper, cobalt, indium, nickel and mixtures thereof. In some embodiments, the nanoparticle core comprises gold. For example, the nanoparticle core can be a lattice structure including degradable gold. Nanoparticles can also comprise semiconductor and magnetic materials.

Non-limiting examples of nanoparticles compatible with aspects of the invention are described in and incorporated by reference from: U.S. Pat. No. 7,238,472, US Patent Publication No. 2003/0147966, US Patent Publication No. 2008/0306016, US Patent Publication No. 2009/0209629, US Patent Publication No. 2010/0136682, US Patent Publication No. 2010/0184844, US Patent Publication No. 2010/0294952, US Patent Publication No. 2010/0129808, US Patent Publication No. 2010/0233270, US Patent Publication No. 2011/0111974, PCT Publication No. WO 2002/096262, PCT Publication No. WO 2003/08539, PCT Publication No. WO 2006/138145, PCT Publication No. WO 2008/127789, PCT Publication No. WO 2008/098248, PCT Publication No. WO 2011/079290, PCT Publication No. WO 2011/053940, PCT Publication No. WO 2011/017690 and PCT Publication No. WO 2011/017456. Nanoparticles associated with the invention can be synthesized according to any means known in the art or can be obtained commercially. For example, several non-limiting examples of commercial suppliers of nanoparticles include: Ted Pella, Inc., Redding, CA, Nanoprobes, Inc., Yaphank, NY, Vacuum Metallurgical Co, Ltd., Chiba, Japan and Vector Laboratories, Inc., Burlington, CA.

### Agonists of Nucleic Acid-Interacting Complexes

A nucleic acid-interacting complex as used herein refers to a molecule or complex of molecules that interact with a nucleic acid molecule and are stimulated to produce an immune response in response to that interaction. The molecule or complex of molecules may be a receptor, for instance. In some embodiments a nucleic acid-interacting complex is a pattern recognition receptor (PRR) complex. PRRs are a primitive part of the immune system composed of proteins expressed by cells of the innate immune system to identify pathogen-associated molecular patterns (PAMPs), which are associated with microbial pathogens or cellular stress, as well as damage-associated molecular patterns (DAMPs), which are associated with cell components released during cell damage. PRRs include but are not limited to membrane-bound PRRs, such as receptor kinases, toll-like receptors (TLR), and C-type lectin Receptors (CLR) (mannose receptors and asialoglycoprotein receptors); Cytoplasmic PRRs such as RIG-I-like receptors (RLR), RNA Helicases, Plant PRRs, and NonRD kinases; and secreted PRRs.

Nucleic acid-interacting complexes include but are not limited to TLRs, RIG-I, transcription factors, cellular translation machinery, cellular transcription machinery, nucleic-acid acting enzymes, and nucleic acid associating autoantigens. Nucleic acid molecules that are agonists of a nucleic acid-interacting complex include but are not limited to TLR agonists, and agonists of RIG-I, transcription factors, cellular translation machinery, cellular transcription machinery, nucleic-acid acting enzymes, and nucleic acid associating autoantigens.

In some embodiments an agonist of a nucleic acid-interacting complex is a TLR agonist. A TLR agonist, as used herein is a nucleic acid molecule that interacts with and stimulates the activity of a TLR.

Toll-like receptors (TLRs) are a family of highly conserved polypeptides that play a critical role in innate immunity in mammals. At least ten family members, designated TLR1-TLR10, have been identified. The cytoplasmic domains of the various TLRs are characterized by a Toll-interleukin 1 (IL-1) receptor (TIR) domain. Medzhitov R et al. (1998) Mol Cell 2:253-8. Recognition of microbial invasion by TLRs triggers activation of a signaling cascade that is evolutionarily conserved in Drosophila and mammals. The TIR domain-containing adaptor protein MyD88 has been reported to associate with TLRs and to recruit IL-1 receptor-associated kinase (IRAK) and tumor necrosis factor (TNF) receptor-associated factor 6 (TRAF6) to the TLRs. The MyD88-dependent signaling pathway is believed to lead to activation of NF-κB transcription factors and c-Jun NH₂ terminal kinase (Jnk) mitogen-activated protein kinases (MAPKs), critical steps in immune activation and production of inflammatory cytokines. For a review, see Aderem A et al. (2000) Nature 406:782-87.

TLRs are believed to be differentially expressed in various tissues and on various types of immune cells. For example, human TLR7 has been reported to be expressed in placenta, lung, spleen, lymph nodes, tonsil and on plasmacytoid precursor dendritic cells (pDCs). Chuang T-H et al. (2000) Eur Cytokine Netw 11:372-8); Kadowaki N et al. (2001) J Exp Med 194:863-9. Human TLR8 has been reported to be expressed in lung, peripheral blood leukocytes (PBL), placenta, spleen, lymph nodes, and on monocytes. Kadowaki N et al. (2001) J Exp Med 194:863-9; Chuang T-H et al. (2000) Eur Cytokine Netw 11:372-8. Human TLR9 is reportedly expressed in spleen, lymph nodes, bone marrow, PBL, and on pDCs, and B cells. Kadowaki N et al. (2001) J Exp Med 194:863-9; Bauer S et al. (2001) Proc Natl Acad Sci USA 98:9237-42; Chuang T-H et al. (2000) Eur Cytokine Netw 11:372-8.

Nucleotide and amino acid sequences of human and murine TLR7 are known. See, for example, GenBank Accession Nos. AF240467, AF245702, NM_016562, AF334942, NM_133211; and AAF60188, AAF78035, NP_057646, AAL73191, and AAL73192, the contents of all of which are incorporated herein by reference. Human TLR7 is reported to be 1049 amino acids long. Murine TLR7 is reported to be 1050 amino acids long. TLR7 polypeptides include an extracellular domain having a leucine-rich repeat region, a transmembrane domain, and an intracellular domain that includes a TIR domain.

Nucleotide and amino acid sequences of human and murine TLR8 are known. See, for example, GenBank Accession Nos. AF246971, AF245703, NM_016610, XM_045706, AY035890, NM_133212; and AAF64061, AAF78036, NP_057694, XP_045706, AAK62677, and NP_573475, the contents of all of which is incorporated herein by reference. Human TLR is reported to exist in at least two isoforms, one 1041 amino acids long and the other 1059 amino acids long. Murine TLR8 is 1032 amino acids long. TLR8 polypeptides include an extracellular domain having a leucine-rich repeat region, a transmembrane domain, and an intracellular domain that includes a TIR domain.

Nucleotide and amino acid sequences of human and murine TLR9 are known. See, for example, GenBank Accession Nos. NM_017442, AF259262, AB045180, AF245704, AB045181, AF348140, AF314224, NM_031178; and NP_059138, AAF72189, BAB19259, AAF78037, BAB19260, AAK29625, AAK28488, and NP_112455, the contents of all of which are incorporated herein by reference. Human TLR9 is reported to exist in at least two isoforms, one 1032 amino acids long and the other 1055 amino acids. Murine TLR9 is 1032 amino acids long. TLR9 polypeptides include an extracellular domain having a leucine-rich repeat region, a transmembrane domain, and an intracellular domain that includes a TIR domain.

As used herein, the term “TLR signaling” refers to any aspect of intracellular signaling associated with signaling through a TLR. As used herein, the term “TLR-mediated immune response” refers to the immune response that is associated with TLR signaling.

A TLR7-mediated immune response is a response associated with TLR7 signaling. TLR7-mediated immune response is generally characterized by the induction of IFN-α and IFN-inducible cytokines such as IP-10 and I-TAC. The levels of cytokines IL-1 α/β, IL-6, IL-8, MIP-1α/β and MIP-3α/β induced in a TLR7-mediated immune response are less than those induced in a TLR8-mediated immune response.

A TLR8-mediated immune response is a response associated with TLR8 signaling. This response is further characterized by the induction of pro-inflammatory cytokines such as IFN-γ, IL-12p40/70, TNF-α, IL-1α/β, IL-6, IL-8, MIP-1 α/β and MIP-3 α/β.

A TLR9-mediated immune response is a response associated with TLR9 signaling. This response is further characterized at least by the production/secretion of IFN-γ and IL-12, albeit at levels lower than are achieved via a TLR8-mediated immune response.

As used herein, a “TLR7/8 agonist” collectively refers to any nucleic acid that is capable of increasing TLR7 and/or TLR8 signaling (i.e., an agonist of TLR7 and/or TLR8). Some TLR7/8 ligands induce TLR7 signaling alone (e.g., TLR7 specific agonists), some induce TLR8 signaling alone (e.g., TLR8 specific agonists), and others induce both TLR7 and TLR8 signaling.

The level of TLR7 or TLR8 signaling may be enhanced over a pre-existing level of signaling or it may be induced over a background level of signaling. TLR7 ligands include, without limitation, guanosine analogues such as C8-substituted guanosines, mixtures of ribonucleosides consisting essentially of G and U, guanosine ribonucleotides and RNA or RNA-like molecules (PCT/US03/10406), and adenosine-based compounds (e.g., 6-amino-9-benzyl-2-(3-hydroxy-propoxy)-9H-purin-8-ol, and similar compounds made by Sumitomo (e.g., CL-029)).

As used herein, the term “guanosine analogues” refers to a guanosine-like nucleotide (excluding guanosine) having a chemical modification involving the guanine base, guanosine nucleoside sugar, or both the guanine base and the guanosine nucleoside sugar. Guanosine analogues specifically include, without limitation, 7-deaza-guanosine.

Guanosine analogues further include C8-substituted guanosines such as 7-thia-8-oxoguanosine (immunosine), 8-mercaptoguanosine, 8-bromoguanosine, 8-methylguanosine, 8-oxo-7,8-dihydroguanosine, C8-arylamino-2′-deoxyguanosine, C8-propynyl-guanosine, C8- and N7-substituted guanine ribonucleosides such as 7-allyl-8-oxoguanosine (loxoribine) and 7-methyl-8-oxoguanosine, 8-aminoguanosine, 8-hydroxy-2′-deoxyguanosine, 8-hydroxyguanosine, and 7-deaza-substituted guanosine.

TLR8 ligands include mixtures of ribonucleosides consisting essentially of G and U, guanosine ribonucleotides and RNA or RNA-like molecules (PCT/US03/10406). Additional TLR8 ligands are also disclosed in Gorden et al. J. Immunol. 2005, 174:1259-1268).

As used herein, the term “TLR9 agonist” refers to any agent that is capable of increasing TLR9 signaling (i.e., an agonist of TLR9). TLR9 agonists specifically include, without limitation, immunostimulatory nucleic acids, and in particular CpG immunostimulatory nucleic acids.

As used herein, the term “immunostimulatory CpG nucleic acids” or “immunostimulatory CpG oligonucleotides” refers to any CpG-containing nucleic acid that is capable of activating an immune cell. At least the C of the CpG dinucleotide is typically, but not necessarily, unmethylated. Immunostimulatory CpG nucleic acids are described in a number of issued patents and published patent applications, including U.S. Pat. Nos. 6,194,388; 6,207,646; 6,218,371; 6,239,116; 6,339,068; 6,406,705; and 6,429,199.

In some embodiments the agonists of nucleic acid-interacting complexes is an immunostimulatory oligonucleotide. An “immunostimulatory oligonucleotide” as used herein is any nucleic acid (DNA or RNA) containing an immunostimulatory motif or backbone that is capable of inducing an immune response. An induction of an immune response refers to any increase in number or activity of an immune cell, or an increase in expression or absolute levels of an immune factor, such as a cytokine. Immune cells include, but are not limited to, NK cells, CD4+ T lymphocytes, CD8+ T lymphocytes, B cells, dendritic cells, macrophage and other antigen-presenting cells. Cytokines include, but are not limited to, interleukins, TNF-α, IFN-α,β and γ, Flt-ligand, and co-stimulatory molecules. Immunostimulatory motifs include, but are not limited to CpG motifs and T-rich motifs.

A non-limiting set of immunostimulatory oligonucleotides includes:

dsRNA (TLR 3): poly(A:C) and poly(I:C)
ssRNA (TLR7/8):
(SEQ ID NO: 13)
CCGUCUGUUGUGUGACUC
(SEQ ID NO: 14)
GCCACCGAGCCGAAGGCACC
(SEQ ID NO: 15)
UAUAUAUAUAUAUAUAUAUA
(SEQ ID NO: 16)
UUAUUAUUAUUAUUAUUAUU
(SEQ ID NO: 17)
UUUUAUUUUAUUUUAUUUUA
(SEQ ID NO: 18)
UGUGUGUGUGUGUGUGUGUG
(SEQ ID NO: 19)
UUGUUGUUGUUGUUGUUGUU
(SEQ ID NO: 20)
UUUGUUUGUUUGUUUGUUUG
(SEQ ID NO: 21)
UUAUUUAUUUAUUUAUUUAUUUAU
(SEQ ID NO: 22)
UUGUUUGUUUGUUUGUUUGUUUGU
(SEQ ID NO: 23)
GCCCGUCUGUUGUGUGACUC
(SEQ ID NO: 24)
GUCCUUCAAGUCCUUCAA
DNA (TLR9):
(SEQ ID NO: 25)
GGTGCATCGATGCAGGGGGG
(SEQ ID NO: 26)
TCCATGGACGTTCCTGAGCGTT
(SEQ ID NO: 27)
TCGTCGTTCGAACGACGTTGAT
(SEQ ID NO: 28)
TCGTCGACGATCCGCGCGCGCG
(SEQ ID NO: 29)
GGGGTCAACGTTGAGGGGGG
(SEQ ID NO: 30)
TCGTCGTTTTGTCGTTTTGTCGTT
(SEQ ID NO: 31)
TCGTCGTTGTCGTTTTGTCGTT
(SEQ ID NO: 32)
GGGGGACGATCGTCGGGGGG
(SEQ ID NO: 33)
GGGGACGACGTCGTGGGGGGG
(SEQ ID NO: 34)
TCGTCGTTTTCGGCGCGCGCCG
(SEQ ID NO: 35)
TCGTCGTCGTTCGAACGACGTTGAT

The immunostimulatory oligonucleotides may be linked to the core or to one another or to other molecules such an antigens. For instance, the oligonucleotides may be conjugated to a linker via the 5′ end or the 3′ end. E.g. [Sequence, 5′-3′]-Linker or Linker-[Sequence, 5′-3′].

The terms “oligonucleotide” and “nucleic acid” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymidine (T) or uracil (U)) or a substituted purine (e.g., adenine (A) or guanine (G)). Thus, the term embraces both DNA and RNA oligonucleotides. The terms shall also include polynucleosides (i.e., a polynucleotide minus the phosphate) and any other organic base containing polymer. Oligonucleotides can be obtained from existing nucleic acid sources (e.g., genomic or cDNA), but are preferably synthetic (e.g., produced by nucleic acid synthesis). A polynucleotide of the nanoscale construct and optionally attached to a nanoparticle core can be single stranded or double stranded. A double stranded polynucleotide is also referred to herein as a duplex. Double-stranded oligonucleotides of the invention can comprise two separate complementary nucleic acid strands.

As used herein, “duplex” includes a double-stranded nucleic acid molecule(s) in which complementary sequences are hydrogen bonded to each other. The complementary sequences can include a sense strand and an antisense strand. The antisense nucleotide sequence can be identical or sufficiently identical to the target gene to mediate effective target gene inhibition (e.g., at least about 98% identical, 96% identical, 94%, 90% identical, 85% identical, or 80% identical) to the target gene sequence.

A double-stranded polynucleotide can be double-stranded over its entire length, meaning it has no overhanging single-stranded sequences and is thus blunt-ended. In other embodiments, the two strands of the double-stranded polynucleotide can have different lengths producing one or more single-stranded overhangs. A double-stranded polynucleotide of the invention can contain mismatches and/or loops or bulges. In some embodiments, it is double-stranded over at least about 70%, 80%, 90%, 95%, 96%, 97%, 98% or 99% of the length of the oligonucleotide. In some embodiments, the double-stranded polynucleotide of the invention contains at least or up to 1, 2, 3, 4, 5, 6, 7, 8, 9, 10, 11, 12, 13, 14, or 15 mismatches.

Polynucleotides associated with the invention can be modified such as at the sugar moiety, the phosphodiester linkage, and/or the base. As used herein, “sugar moieties” includes natural, unmodified sugars, including pentose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol.

Modification of sugar moieties can include 2′-O-methyl nucleotides, which are referred to as “methylated.” In some instances, polynucleotides associated with the invention may only contain modified or unmodified sugar moieties, while in other instances, polynucleotides contain some sugar moieties that are modified and some that are not.

In some instances, modified nucleomonomers include sugar- or backbone-modified ribonucleotides. Modified ribonucleotides can contain a non-naturally occurring base such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides can have the 2′-OH group replaced by an H, alkoxy (or OR), R or alkyl, halogen, SH, SR, amino (such as NH₂, NHR, NR_{2,}), or CN group, wherein R is lower alkyl, alkenyl, or alkynyl. In some embodiments, modified ribonucleotides can have the phosphodiester group connecting to adjacent ribonucleotides replaced by a modified group, such as a phosphorothioate group.

In some aspects, 2′-O-methyl modifications can be beneficial for reducing undesirable cellular stress responses, such as the interferon response to double-stranded nucleic acids. Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano and the like. The sugar moiety can also be a hexose.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In some embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids. The term “n-alkyl” means a straight chain (i.e., unbranched) unsubstituted alkyl group.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In some embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Unless otherwise specified, the term alkenyl includes both “unsubstituted alkenyls” and “substituted alkenyls,” the latter of which refers to alkenyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “hydrophobic modifications’ refers to modification of bases such that overall hydrophobicity is increased and the base is still capable of forming close to regular Watson-Crick interactions. Non-limiting examples of base modifications include 5-position uridine and cytidine modifications like phenyl, 4-pyridyl, 2-pyridyl, indolyl, and isobutyl, phenyl (C₆H₅OH); tryptophanyl (C₈H₆N)CH₂CH(NH₂)CO), Isobutyl, butyl, aminobenzyl; phenyl; naphthyl,

The term “heteroatom” includes atoms of any element other than carbon or hydrogen. In some embodiments, preferred heteroatoms are nitrogen, oxygen, sulfur and phosphorus. The term “hydroxy” or “hydroxyl” includes groups with an —OH or —O⁻ (with an appropriate counterion). The term “halogen” includes fluorine, bromine, chlorine, iodine, etc. The term “perhalogenated” generally refers to a moiety wherein all hydrogens are replaced by halogen atoms.

The term “substituted” includes independently selected substituents which can be placed on the moiety and which allow the molecule to perform its intended function. Examples of substituents include alkyl, alkenyl, alkynyl, aryl, (CR′R″)₀₋₃NR′R″, (CR′R″)₀₋₃CN, NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂R′, or (CR′R″)₀₋₃OR′ groups; wherein each R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

The term “amine” or “amino” includes compounds or moieties in which a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups.

The term “ether” includes compounds or moieties which contain an oxygen bonded to two different carbon atoms or heteroatoms. For example, the term includes “alkoxyalkyl,” which refers to an alkyl, alkenyl, or alkynyl group covalently bonded to an oxygen atom which is covalently bonded to another alkyl group.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N⁶-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In some aspects, the nucleomonomers of a polynucleotide of the invention are RNA nucleotides, including modified RNA nucleotides.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2^{nd} Ed., Wiley-Interscience, New York, 1999).

The term “nucleotide” includes nucleosides which further comprise a phosphate group or a phosphate analog.

As used herein, the term “linkage” includes a naturally occurring, unmodified phosphodiester moiety (—O—(PO²⁻)—O—) that covalently couples adjacent nucleomonomers. As used herein, the term “substitute linkage” includes any analog or derivative of the native phosphodiester group that covalently couples adjacent nucleomonomers. Substitute linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47). In certain embodiments, non-hydrolizable linkages are preferred, such as phosphorothioate linkages.

In some aspects, polynucleotides of the invention comprise 3′ and 5′ termini (except for circular oligonucleotides). The 3′ and 5′ termini of a polynucleotide can be substantially protected from nucleases, for example, by modifying the 3′ or 5′ linkages (e.g., U.S. Pat. No. 5,849,902 and WO 98/13526). Oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²⁻), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl can be esterified to a nucleotide through a 3′-+3′ internucleotide linkage. For example, the alkyloxy radical can be methoxy, ethoxy, or isopropoxy, and preferably, ethoxy. Optionally, the 3′→3′ linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

In some aspects, polynucleotides can comprise both DNA and RNA.

In some aspects, at least a portion of the contiguous polynucleotides are linked by a substitute linkage, e.g., a phosphorothioate linkage. The presence of substitute linkages can improve pharmacokinetics due to their higher affinity for serum proteins.

CpG sequences, while relatively rare in human DNA are commonly found in the DNA of infectious organisms such as bacteria. The human immune system has apparently evolved to recognize CpG sequences as an early warning sign of infection and to initiate an immediate and powerful immune response against invading pathogens without causing adverse reactions frequently seen with other immune stimulatory agents. Thus CpG containing nucleic acids, relying on this innate immune defense mechanism can utilize a unique and natural pathway for immune therapy. The effects of CpG nucleic acids on immune modulation have been described extensively in U.S. Pat. No. 6,194,388, and published patent applications, such as PCT US95/01570), PCT/US97/19791, PCT/US98/03678; PCT/US98/10408; PCT/US98/04703; PCT/US99/07335; and PCT/US99/09863.

A “CpG oligonucleotide” is a nucleic acid which includes at least one unmethylated CpG dinucleotide. In some embodiments, the nucleic acid includes three or more unmethylated CpG dinucleotides. A nucleic acid containing at least one “unmethylated CpG dinucleotide” is a nucleic acid molecule which contains an unmethylated cytosine in a cytosine-guanine dinucleotide sequence (i.e. “CpG DNA” or DNA containing a 5′ cytosine followed by 3′ guanosine and linked by a phosphate bond) and activates the immune system.

The immunostimulatory oligonucleotides of the nanoscale construct are preferably in the range of 6 to 100 bases in length. However, nucleic acids of any size greater than 6 nucleotides (even many kb long) are capable of inducing an immune response according to the invention if sufficient immunostimulatory motifs are present. Preferably the immunostimulatory nucleic acid is in the range of between 8 and 100 and in some embodiments between 8 and 50 or 8 and 30 nucleotides in size.

In some embodiments the immunostimulatory oligonucleotides have a modified backbone such as a phosphorothioate (PS) backbone. In other embodiments the immunostimulatory oligonucleotides have a phosphodiester (PO) backbone. In yet other embodiments immunostimulatory oligonucleotides have a mixed PO and PS backbone.

### Attachment of Modalities to Nanoparticle Cores

Modalities associated with the invention, including agonists of nucleic acid-interacting complexes and antigens, can be attached to nanoparticle cores by any means known in the art. Methods for attaching oligonucleotides to nanoparticles are described in detail in and incorporated by reference from US Patent Publication No. 2010/0129808.

A nanoparticle can be functionalized in order to attach a polynucleotide. Alternatively or additionally, the polynucleotide can be functionalized. One mechanism for functionalization is the alkanethiol method, whereby oligonucleotides are functionalized with alkanethiols at their 3′ or 5′ termini prior to attachment to gold nanoparticles or nanoparticles comprising other metals, semiconductors or magnetic materials. Such methods are described, for example Whitesides, Proceedings of the Robert A. Welch Foundation 39th Conference On Chemical Research Nanophase Chemistry, Houston, Tex., pages 109-121 (1995), and Mucic et al. Chem. Commun. 555-557 (1996). Oligonucleotides can also be attached to nanoparticles using other functional groups such as phosphorothioate groups, as described in and incorporated by reference from U.S. Pat. No. 5,472,881, or substituted alkylsiloxanes, as described in and incorporated by reference from Burwell, Chemical Technology, 4, 370-377 (1974) and Matteucci and Caruthers, J. Am. Chem. Soc., 103, 3185-3191 (1981). In some instances, polynucleotides are attached to nanoparticles by terminating the polynucleotide with a 5′ or 3′ thionucleoside. In other instances, an aging process is used to attach polynucleotides to nanoparticles as described in and incorporated by reference from U.S. Pat. Nos. 6,361,944, 6,506,569, 6,767,702 and 6,750,016 and PCT Publication Nos. WO 1998/004740, WO 2001/000876, WO 2001/051665 and WO 2001/073123.

In some instances, the nucleic acid and/or antigen are covalently attached to the nanoparticle core, such as through a gold-thiol linkage. A spacer sequence can be included between the attachment site and the uptake control moiety and/or the binding moiety. In some embodiments, a spacer sequence comprises or consists of an oligonucleotide, a peptide, a polymer or an oligoethylene.

Nanoscale constructs can be designed with multiple chemistries. For example, a DTPA (dithiol phosphoramidite) linkage can be used. The DTPA resists intracellular release of flares by thiols and can serve to increase signal to noise ratio.

The conjugates produced by the methods described herein are considerably more stable than those produced by other methods. This increased stability is due to the increased density of the oligonucleotides on the surfaces of a nanoparticle core or forming the surface of the corona. By performing the salt additions in the presence of a surfactant, for example approximately 0.01% sodium dodecylsulfate (SDS), Tween, or polyethylene glycol (PEG), the salt aging process can be performed in about an hour.

The surface density may depend on the size and type of nanoparticles and on the length, sequence and concentration of the oligonucleotides. A surface density adequate to make the nanoparticles stable and the conditions necessary to obtain it for a desired combination of nanoparticles and oligonucleotides can be determined empirically. Generally, a surface density of at least 10 picomoles/cm will be adequate to provide stable nanoparticle-oligonucleotide conjugates. Preferably, the surface density is at least 15 picomoles/cm. Since the ability of the oligonucleotides of the conjugates to hybridize with targets may be diminished if the surface density is too great, the surface density optionally is no greater than about 35-40 picomoles/cm². Methods are also provided wherein the oligonucleotide is bound to the nanoparticle at a surface density of at least 10 pmol/cm², at least 15 pmol/cm², at least 20 pmol/cm², at least 25 pmol/cm², at least 30 pmol/cm², at least 35 pmol/cm², at least 40 pmol/cm², at least 45 pmol/cm, at least 50 pmol/cm², or 50 pmol/cm² or more.

### Therapeutics

Aspects of the invention relate to delivery of nanoscale constructs to a subject for therapeutic and/or diagnostic use. The particles may be administered alone or in any appropriate pharmaceutical carrier, such as a liquid, for example saline, or a powder, for administration in vivo. They can also be co-delivered with larger carrier particles or within administration devices. The particles may be formulated. The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients. In some embodiments, nanoscale constructs associated with the invention are mixed with a substance such as a lotion (for example, aquaphor) and are administered to the skin of a subject, whereby the nanoscale constructs are delivered through the skin of the subject. It should be appreciated that any method of delivery of nanoparticles known in the art may be compatible with aspects of the invention.

For use in therapy, an effective amount of the particles can be administered to a subject by any mode that delivers the particles to the desired cell. Administering pharmaceutical compositions may be accomplished by any means known to the skilled artisan. Routes of administration include but are not limited to oral, parenteral, intramuscular, intravenous, subcutaneous, mucosal, intranasal, sublingual, intratracheal, inhalation, ocular, vaginal, dermal, rectal, and by direct injection.

Thus, the invention in one aspect involves the finding that agonists of nucleic acid-interacting complexes are highly effective in mediating immune stimulatory effects. These agonists of nucleic acid-interacting complexes are useful therapeutically and prophylactically for stimulating the immune system to treat cancer, infectious diseases, allergy, asthma, autoimmune disease, and other disorders and to help protect against opportunistic infections following cancer chemotherapy. The strong yet balanced, cellular and humoral immune responses that result from, for example, TLR agonist stimulation reflect the body's own natural defense system against invading pathogens and cancerous cells.

Thus the agonists of nucleic acid-interacting complexes useful in some aspects of the invention as a vaccine for the treatment of a subject at risk of developing or a subject having allergy or asthma, an infection with an infectious organism or a cancer in which a specific cancer antigen has been identified. The agonists of nucleic acid-interacting complexes can also be given without the antigen or allergen for protection against infection, allergy or cancer, and in this case repeated doses may allow longer term protection. A subject at risk as used herein is a subject who has any risk of exposure to an infection causing pathogen or a cancer or an allergen or a risk of developing cancer. For instance, a subject at risk may be a subject who is planning to travel to an area where a particular type of infectious agent is found or it may be a subject who through lifestyle or medical procedures is exposed to bodily fluids which may contain infectious organisms or directly to the organism or even any subject living in an area where an infectious organism or an allergen has been identified. Subjects at risk of developing infection also include general populations to which a medical agency recommends vaccination with a particular infectious organism antigen. If the antigen is an allergen and the subject develops allergic responses to that particular antigen and the subject may be exposed to the antigen, i.e., during pollen season, then that subject is at risk of exposure to the antigen.

A subject having an infection is a subject that has been exposed to an infectious pathogen and has acute or chronic detectable levels of the pathogen in the body. The CpG immunostimulatory oligonucleotides can be used with or without an antigen to mount an antigen specific systemic or mucosal immune response that is capable of reducing the level of or eradicating the infectious pathogen. An infectious disease, as used herein, is a disease arising from the presence of a foreign microorganism in the body. It is particularly important to develop effective vaccine strategies and treatments to protect the body's mucosal surfaces, which are the primary site of pathogenic entry.

A subject having an allergy is a subject that has or is at risk of developing an allergic reaction in response to an allergen. An allergy refers to acquired hypersensitivity to a substance (allergen). Allergic conditions include but are not limited to eczema, allergic rhinitis or coryza, hay fever, conjunctivitis, bronchial asthma, urticaria (hives) and food allergies, and other atopic conditions.

A subject having a cancer is a subject that has detectable cancerous cells. The cancer may be a malignant or non-malignant cancer. Cancers or tumors include but are not limited to biliary tract cancer, brain cancer, breast cancer; cervical cancer, choriocarcinoma; colon cancer; endometrial cancer, esophageal cancer, gastric cancer; intraepithelial neoplasms; lymphomas; liver cancer, lung cancer (e.g. small cell and non-small cell); melanoma; neuroblastomas; oral cancer; ovarian cancer, pancreas cancer; prostate cancer; rectal cancer; sarcomas; skin cancer; testicular cancer; thyroid cancer; and renal cancer, as well as other carcinomas and sarcomas. In one embodiment the cancer is hairy cell leukemia, chronic myelogenous leukemia, cutaneous T-cell leukemia, multiple myeloma, follicular lymphoma, malignant melanoma, squamous cell carcinoma, renal cell carcinoma, prostate carcinoma, bladder cell carcinoma, or colon carcinoma.

A subject shall mean a human or vertebrate animal including but not limited to a dog, cat, horse, cow, pig, sheep, goat, turkey, chicken, primate, e.g., monkey, and fish (aquaculture species), e.g. salmon. Thus, the invention can also be used to treat cancer and tumors, infections, and allergy/asthma in non human subjects.

As used herein, the term treat, treated, or treating when used with respect to an disorder such as an infectious disease, cancer, allergy, or asthma refers to a prophylactic treatment which increases the resistance of a subject to development of the disease (e.g., to infection with a pathogen) or, in other words, decreases the likelihood that the subject will develop the disease (e.g., become infected with the pathogen) as well as a treatment after the subject has developed the disease in order to fight the disease (e.g., reduce or eliminate the infection) or prevent the disease from becoming worse.

An antigen as used herein is a molecule capable of provoking an immune response. Antigens include but are not limited to cells, cell extracts, proteins, polypeptides, peptides, polysaccharides, polysaccharide conjugates, peptide and non-peptide mimics of polysaccharides and other molecules, small molecules, lipids, glycolipids, carbohydrates, viruses and viral extracts and multicellular organisms such as parasites and allergens. The term antigen broadly includes any type of molecule which is recognized by a host immune system as being foreign. Antigens include but are not limited to cancer antigens, microbial antigens, and allergens.

As used herein, the terms “cancer antigen” and “tumor antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. A cancer antigen is a compound, such as a peptide or protein, associated with a tumor or cancer cell surface and which is capable of provoking an immune response when expressed on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens.

A microbial antigen as used herein is an antigen of a microorganism and includes but is not limited to virus, bacteria, parasites, and fungi. Such antigens include the intact microorganism as well as natural isolates and fragments or derivatives thereof and also synthetic compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art.

Examples of viruses that have been found in humans include but are not limited to: Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Both gram negative and gram positive bacteria serve as antigens in vertebrate animals. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borrelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

Examples of fungi include Cryptococcus neoformans, Histoplasma capsulatum, Coccidioides immitis, Blastomyces dermatitidis, Chlamydia trachomatis, Candida albicans.

Other infectious organisms (i.e., protists) include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovan, Trypanosoma gambiense and Trypanosoma rhodesiense (African sleeping sickness), Trypanosoma cruzi (Chagas' disease), and Toxoplasma gondii.

Other medically relevant microorganisms have been described extensively in the literature, e.g., see C. G. A Thomas, Medical Microbiology, Bailliere Tindall, Great Britain 1983, the entire contents of which is hereby incorporated by reference.

An allergen refers to a substance (antigen) that can induce an allergic or asthmatic response in a susceptible subject. The list of allergens is enormous and can include pollens, insect venoms, animal dander dust, fungal spores and drugs (e.g. penicillin). Examples of natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemisifolia; Lolium (e.g. Lolium perenne or Lolium multiflorum); Cryptomeria (Cryptomeria japonica); Alternaria (Alternaria alternata); Alder; Alnus (Alnus glutinosa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

The nanoscale constructs of the invention may also be coated with or administered in conjunction with an anti-microbial agent. An anti-microbial agent, as used herein, refers to a naturally-occurring or synthetic compound which is capable of killing or inhibiting infectious microorganisms. The type of anti-microbial agent useful according to the invention will depend upon the type of microorganism with which the subject is infected or at risk of becoming infected. Anti-microbial agents include but are not limited to anti-bacterial agents, anti-viral agents, anti-fungal agents and anti-parasitic agents. Phrases such as “anti-infective agent”, “anti-bacterial agent”, “anti-viral agent”, “anti-fungal agent”, “anti-parasitic agent” and “parasiticide” have well-established meanings to those of ordinary skill in the art and are defined in standard medical texts. Briefly, anti-bacterial agents kill or inhibit bacteria, and include antibiotics as well as other synthetic or natural compounds having similar functions. Antibiotics are low molecular weight molecules which are produced as secondary metabolites by cells, such as microorganisms. In general, antibiotics interfere with one or more bacterial functions or structures which are specific for the microorganism and which are not present in host cells. Anti-viral agents can be isolated from natural sources or synthesized and are useful for killing or inhibiting viruses. Anti-fungal agents are used to treat superficial fungal infections as well as opportunistic and primary systemic fungal infections. Anti-parasite agents kill or inhibit parasites.

Antibacterial agents kill or inhibit the growth or function of bacteria. A large class of antibacterial agents is antibiotics. Antibiotics, which are effective for killing or inhibiting a wide range of bacteria, are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics. Antibacterial agents am sometimes classified based on their primary mode of action. In general, antibacterial agents are cell wall synthesis inhibitors, cell membrane inhibitors, protein synthesis inhibitors, nucleic acid synthesis or functional inhibitors, and competitive inhibitors.

Antiviral agents are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There am several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleotide analogues), maturation of new virus proteins (e.g. protease inhibitors), and budding and release of the virus.

The constructs of the invention may also be administered in conjunction with a therapeutic or diagnostic antibody. In one embodiment, the antibody may be selected from the group consisting of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MFLIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab, rituxan, bevacizumab, and ImmuRAIT-CEA.

The agonists of nucleic acid-interacting complexes are also useful for treating and preventing autoimmune disease. Autoimmune disease is a class of diseases in which an subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

A “self-antigen” as used herein refers to an antigen of a normal host tissue. Normal host tissue does not include cancer cells. Thus an immune response mounted against a self-antigen, in the context of an autoimmune disease, is an undesirable immune response and contributes to destruction and damage of normal tissue, whereas an immune response mounted against a cancer antigen is a desirable immune response and contributes to the destruction of the tumor or cancer. Thus, in some aspects of the invention aimed at treating autoimmune disorders it is not recommended that the CpG immunostimulatory nucleic acids be administered with self antigens, particularly those that are the targets of the autoimmune disorder.

In other instances, the CpG immunostimulatory nucleic acids may be delivered with low doses of self-antigens. A number of animal studies have demonstrated that mucosal administration of low doses of antigen can result in a state of immune hyporesponsiveness or “tolerance.” The active mechanism appears to be a cytokine-mediated immune deviation away from a Th1 towards a predominantly Th2 and Th3 (i.e., TGF-β dominated) response. The active suppression with low dose antigen delivery can also suppress an unrelated immune response (bystander suppression) which is of considerable interest in the therapy of autoimmune diseases, for example, rheumatoid arthritis and SLE. Bystander suppression involves the secretion of Th1-counter-regulatory, suppressor cytokines in the local environment where proinflammatory and Th1 cytokines are released in either an antigen-specific or antigen-nonspecific manner. “Tolerance” as used herein is used to refer to this phenomenon. Indeed, oral tolerance has been effective in the treatment of a number of autoimmune diseases in animals including: experimental autoimmune encephalomyelitis (EAE), experimental autoimmune myasthenia gravis, collagen-induced arthritis (CIA), and insulin-dependent diabetes mellitus. In these models, the prevention and suppression of autoimmune disease is associated with a shift in antigen-specific humoral and cellular responses from a Th1 to Th2/Th3 response.

In another aspect, the present invention is directed to a kit including one or more of the compositions previously discussed. A “kit,” as used herein, typically defines a package or an assembly including one or more of the compositions of the invention, and/or other compositions associated with the invention, for example, as previously described. Each of the compositions of the kit, if present, may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions that may be associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, tapes, adhesives, and the like, for example, for using, administering, modifying, assembling, storing, packaging, preparing, mixing, diluting, and/or preserving the compositions components for a particular use, for example, to a sample and/or a subject.

In some embodiments, a kit associated with the invention includes one or more nanoparticle cores, such as a nanoparticle core that comprises gold. A kit can also include one or more agonists of nucleic acid-interacting complexes. A kit can also include one or more antigens.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the use of the compositions, for example, for a particular use, e.g., to a sample. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, the present invention is directed to methods of promoting one or more embodiments of the invention as discussed herein. As used herein, “promoting” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1

### Materials and Methods

### Synthesis of Immunostimulatory SNAs

Synthesis of immunostimulatory SNAs (isSNA) is achieved as described elsewhere⁷⁻¹² with the following essential modifications. In brief, 20 mL 13 nm gold colloid is mixed with 10% Tween 20 and TLR agonist sequence of sulfhydryl-modified nucleic acid (TLR 3, 7/8, 9) at appropriate concentration, such as 5 uM and allowed to react overnight. Addition of antigen can be achieved similar to methods as described elsewhere.¹⁰ Purification of SNAs can be achieved by repeated ultracentrifugation at 75,000×g for 30 min.

### Cell Lines

RAW 264.7 cell lines were obtained from ATCC. RAW-Blue macrophages were obtained from InVivoGen. Ramos-Blue and THP1-XBlue cells were obtained from InVivoGen. All were cultured according to the distributor recommendations.

### Results and Discussion

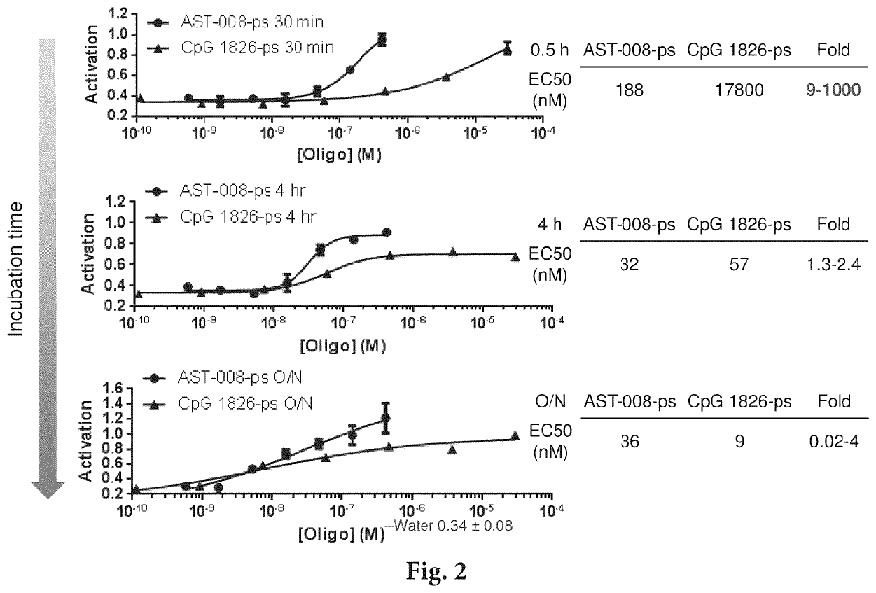

It was found that the nanoscale constructs of the invention markedly enhance potency in macrophages over unformulated agonists of nucleic acid-interacting complexes (CpG oligonucleotides) in solution (FIG. 2). RAW-Blue macrophages were plated at 65 k cells per well and allowed to adhere overnight. On the day of experiments, cells were treated with AST-008-ps or CpG 1826-ps at the indicated concentration of oligo for 30 min (top panel), 4 h (middle panel), or overnight (bottom panel). For 30 min and 4 h time points, the entire supernatant was aspirated, the cells were washed, and complete growth medium without test agents were administered. At the overnight time point, the activation state of the cells was determined using the QuantiBlue assay kit. The results show that particularly at short time points, AST-008-ps demonstrates a significantly lower EC50 than CpG 1826-ps of 188 nM as compared to 17800 nM (9-1000-fold reduction one standard deviation from the mean). At 4 h, AST-008-ps demonstrates a lower EC50 (32 nM vs 57 nm) and greater activation state than CpG 1826-ps. This difference became narrower following overnight incubation, at which point the EC50s were not statistically different from each other. This suggests that under conditions where residence time of the agent with the target cell is limited, particularly to 30 minutes or below, the AST-008-ps formulation may result in more rapid and robust immune activation.

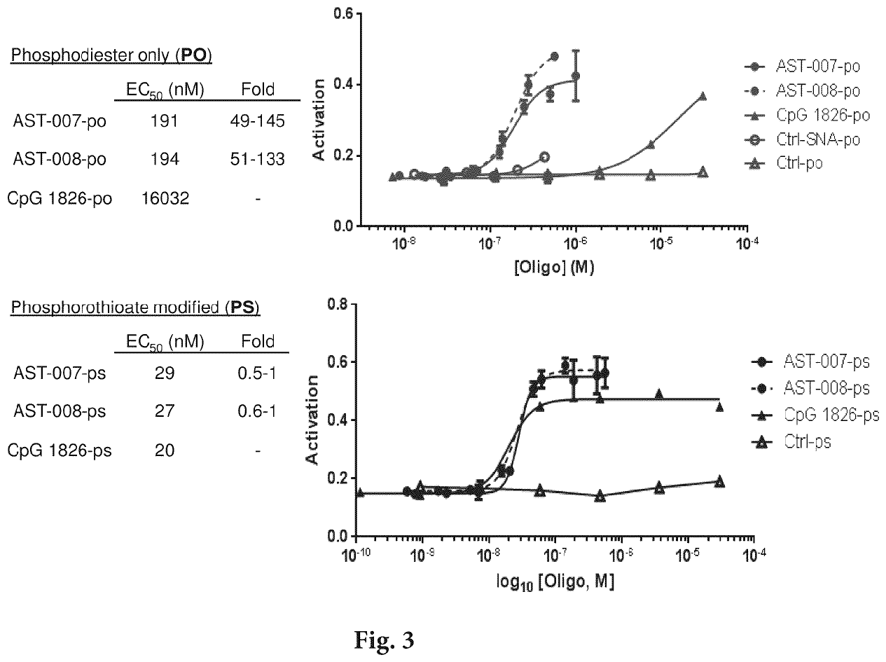

It was also demonstrated that the nanoscale constructs of the invention markedly enhanced potency in macrophages over unformulated agonists of nucleic acid-interacting complexes (CpG oligonucleotides) in solution following overnight incubation (FIG. 3). RAW-Blue macrophages were plated at 65 k cells per well and allowed to adhere overnight. On the day of experiments, AST-007-po, AST-007-ps, AST-008-po, AST-008-ps, CpG 1826-po, CpG 1826-ps were incubated with the cells overnight. The degree of activation was then determined using the QuantiBlue assay kit. The results show that for compounds containing only phosphodiester (-po) linkages (top panel), AST-007-po and AST-008-po are ˜50-150 fold more potent as determined by their EC50 values than CpG 1826-po (191 nM and 194 nM as compared to 16032 nM, respectively). With phosphorothioate-modified compounds (-ps, bottom panel), AST-007-ps and AST-008-ps are approximately equivalent to CpG 1826-ps, with an EC50 of 29 nM, 27 nM, and 20 nM, respectively.

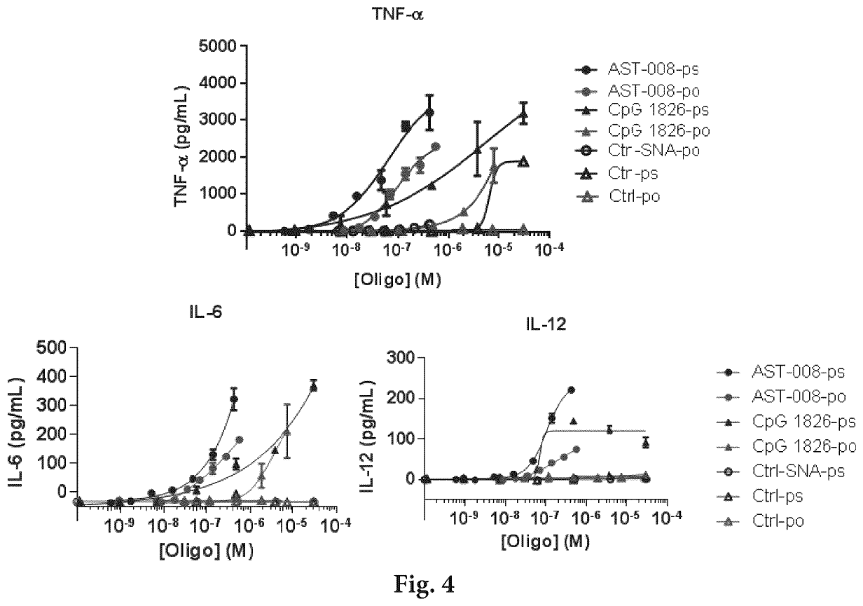

Levels of cytokine secretion following exposure to the nanoscale constructs of the invention versus unformulated agonists of nucleic acid-interacting complexes (CpG oligonucleotides) in solution was examined. The effect on cytokine induction was examined for both oligonucleotides having phosphodiester and phosphorothioate internucleotide linkages in both the nanoparticle and the TRL agonist groups (FIG. 4). RAW-Blue macrophages were plated at 65 k cells per well and allowed to adhere overnight. On the day of experiments, the indicated compounds were incubated with the cells overnight. The degree of cytokine secretion was determined by collecting the supernatant and measuring the concentration of the indicated cytokines by ELISA (TNF-alpha—top panel, IL-12—bottom right panel, IL-6—bottom left panel). The results show significantly higher cytokine secretion at lower doses are possible using AST-008-ps and AST-008-po than CpG 1826-ps, CpG 1826-po, and the indicated controls. For example, to achieve greater than 2000 pg/mL TNF-alpha, less than 100 nM AST-008-ps was needed and less than 1000 nM AST-007-po was needed, but greater than 1000 nM CpG 1826-ps was required.

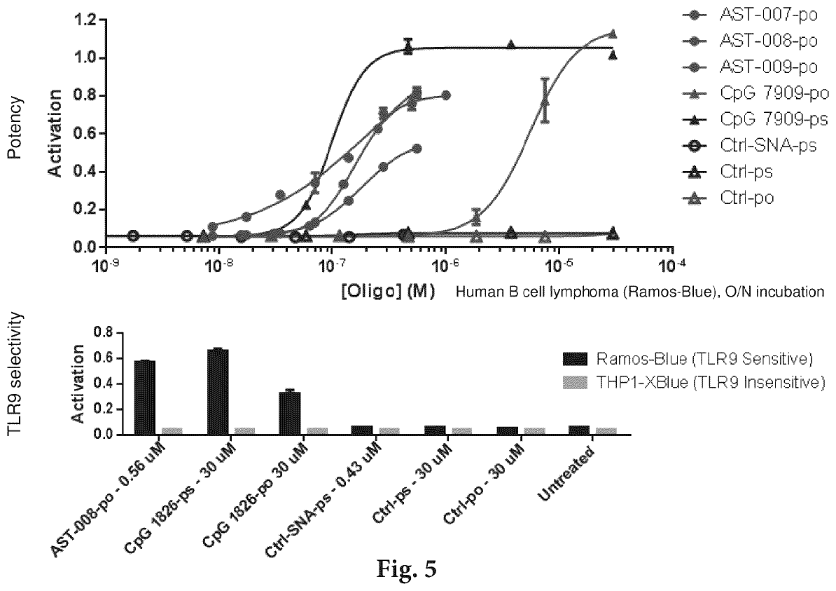

Next, TLR9 activation in response to stimulation with a nanoscale of the invention having a phosphodiester CpG oligonucleotide in comparison with phosphodiester and phosphorothioate CpG oligonucleotides in solution was examined (FIG. 5). Ramos-Blue or THP1-XBlue cells were seeded and activated according to the manufacturer's recommended protocol using the indicated compounds and controls. Remarkably, AST-007-po, AST-008-po, and AST-009-po demonstrate comparable activation at similar doses than CpG 7909-ps, a known and optimized TLR 9 agonist. In addition, activation appeared to be dependent on TLR 9, as the TLR 9 agonist insensitive THP1-XBlue cells did not demonstrate any activation.

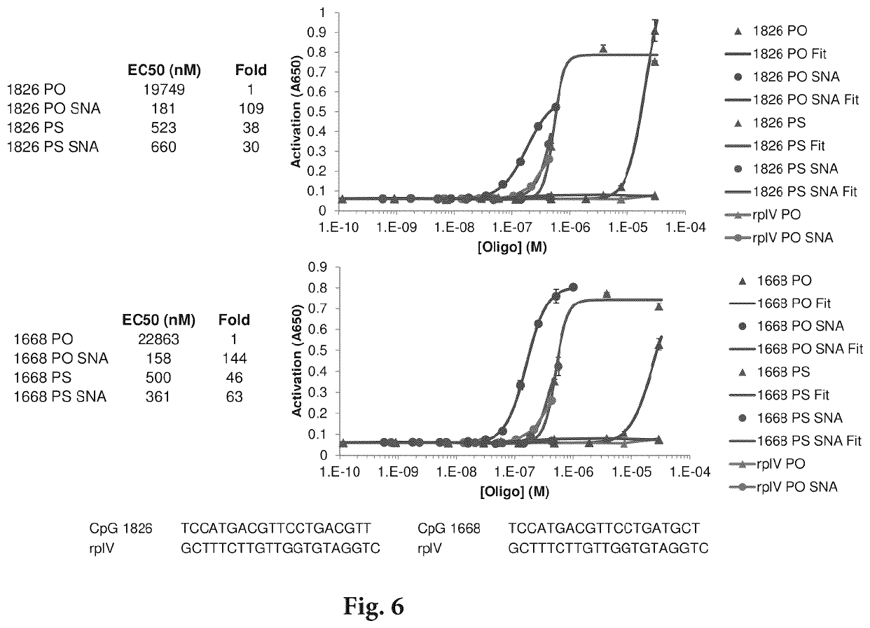

It was determined that a nanoscale construct of the invention had a multiple fold increase in potency of over several different CpG oligo sequences (FIG. 6). Ramos-Blue cells were seeded and activated according to the manufacturer's recommended protocol using the indicated compounds and controls. Oligo 1826 (top panel) and 1668 (bottom panel) were tested. Notably, SNA compounds demonstrate significantly lower EC50 values than free oligos, independent of chemistry, as compared to controls. This suggests that for these sequences, the SNA formulation of oligos is several fold mom potent.

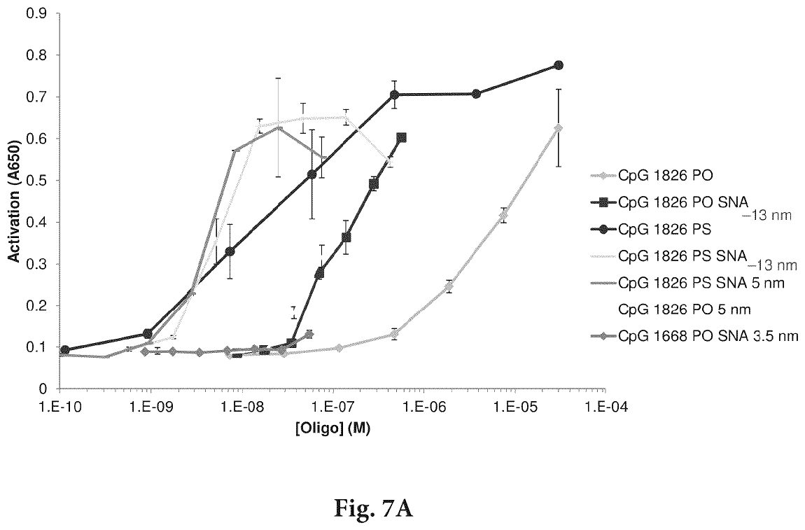

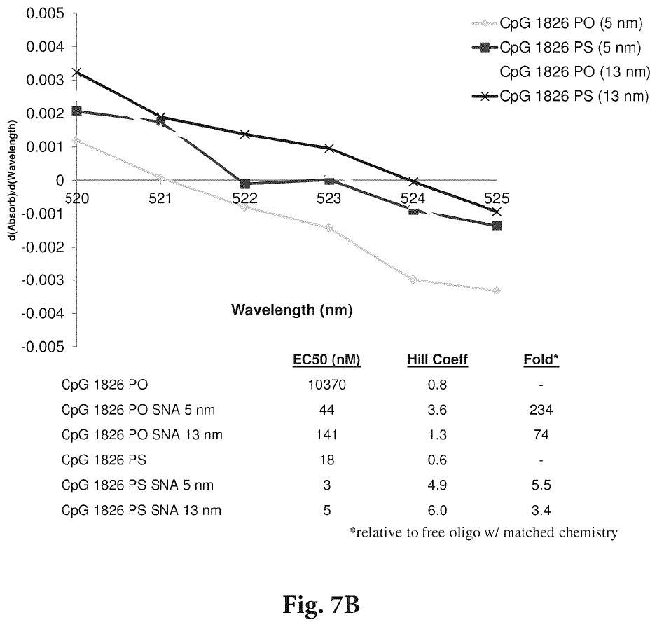

The effects of modulating nanoparticle core size was examined (FIGS. 7A-7B). Raw Blue cells were plated and treated with the indicated agonists with different gold core sizes, ranging from 3.5 nm to 13 nm using the indicated oligos. The results show that smaller gold core sizes appear to demonstrate the potential to enhance agonist activity in vitro.

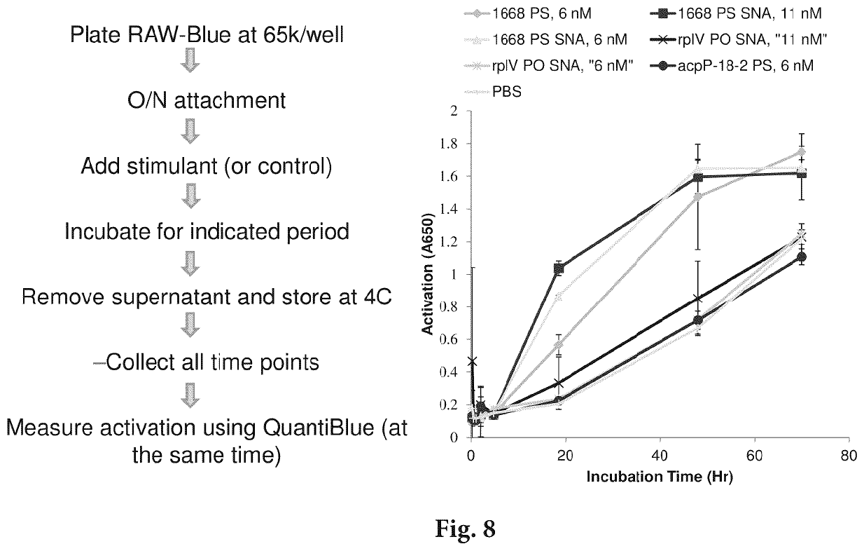

The nanoscale constructs of the invention were observed to have a more rapid and sustained activation than CpG oligo (FIG. 8). Cells were plated as described and activation was measured using QuantiBlue. The results show that at 6 nM oligo, the PS SNAs demonstrate significantly more activation than free 1668 PS oligos.

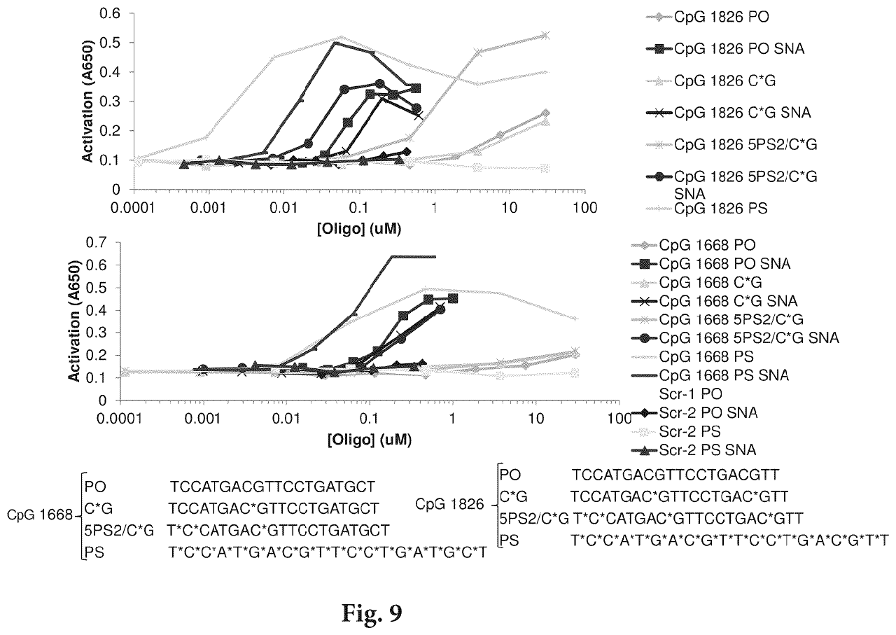

The ability of phosphorothioate modifications to modulate agonist activity in a sequence-dependent manner was examined (FIG. 9). Raw Blue cells were plated and treated with the indicated agonists. Oligo 1826 (top panel) and 1668 (bottom panel) were tested. The results show that internal phosphorothioate modifications (C*G) and two 5′ phosphorothioate linkages (5′PS2) have an effect on the activity of immunostimulatory SNAs that appears to be sequence dependent.

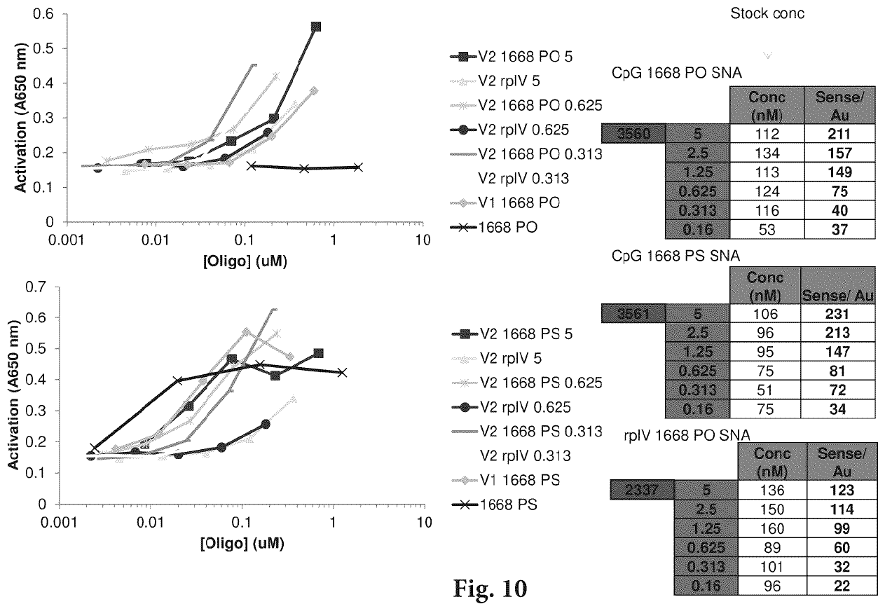

The ability of oligonucleotide loading density to affect agonist activity was assessed (FIG. 10). V2 indicates that the construct was completely gold coated prior to oligo addition to the gold core. Phosphodiester oligonucleotides (top panel) and phosphorothioate oligonucleotides (bottom panel) were tested. The data shows that the density of oligonucleotide on the surface of the gold will modulate the activity of the immunostimulatory construct.

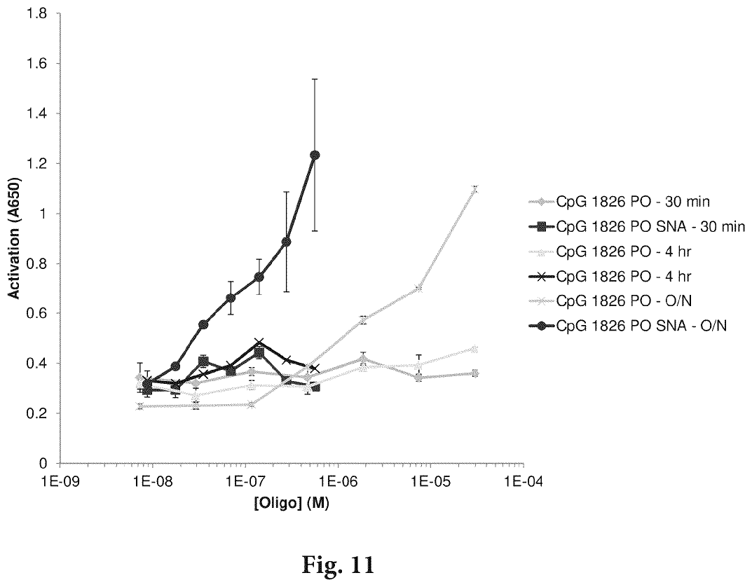

A time course of activation of CpG PO/PO nanoscale constructs was studied (FIG. 11). The tested constructs are not activated until >4 hr of incubation. Raw Blue cells were plated and treated with the indicated agonists. The results show that PO and PO SNAs do not robustly activate adherent RAW Blue cells until greater than 4 hours of incubation.

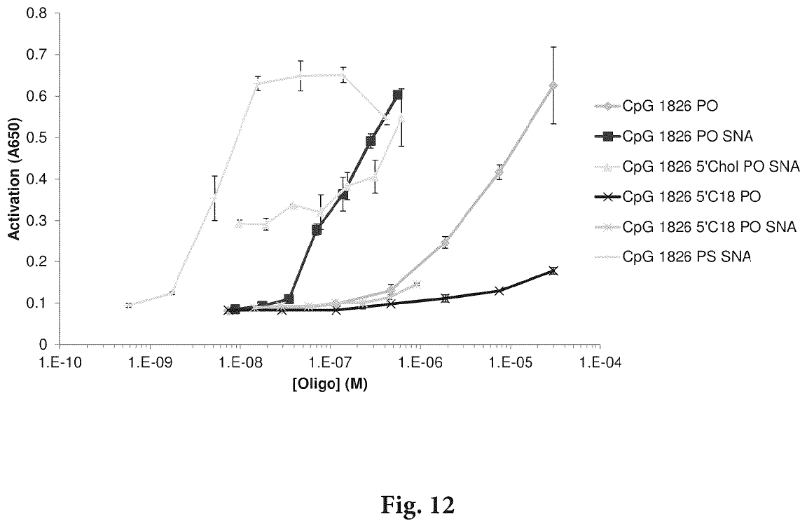

5′Chol CpG PO nanoscale constructs showed activation in low nM range, while 5′C18 abrogated the activity (FIG. 12). A 5′ cholesterol modification (5′Chol) may increase the potency of the agonist, particularly at low concentrations in a oligo-dose-independent manner. Modification of the 5′ end with a C18 molecule (5′C18) appears to eliminate the activity altogether.

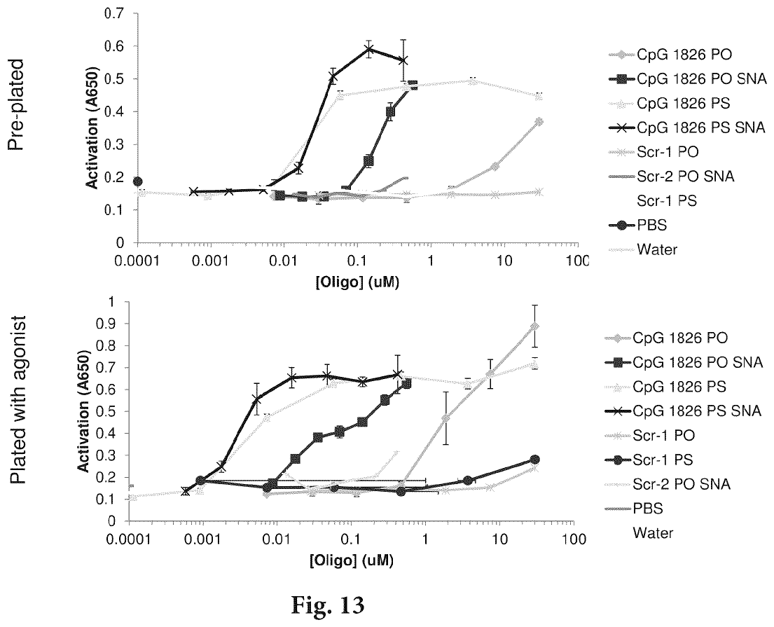

Pre-plated macrophages are more primed for subsequent activation (FIG. 13). RAW Blue macrophages that were plated overnight prior to addition of the agonist compounds (top) generally demonstrate greater activation than when the cells are plated at the same time as the agonist compound is added (bottom).

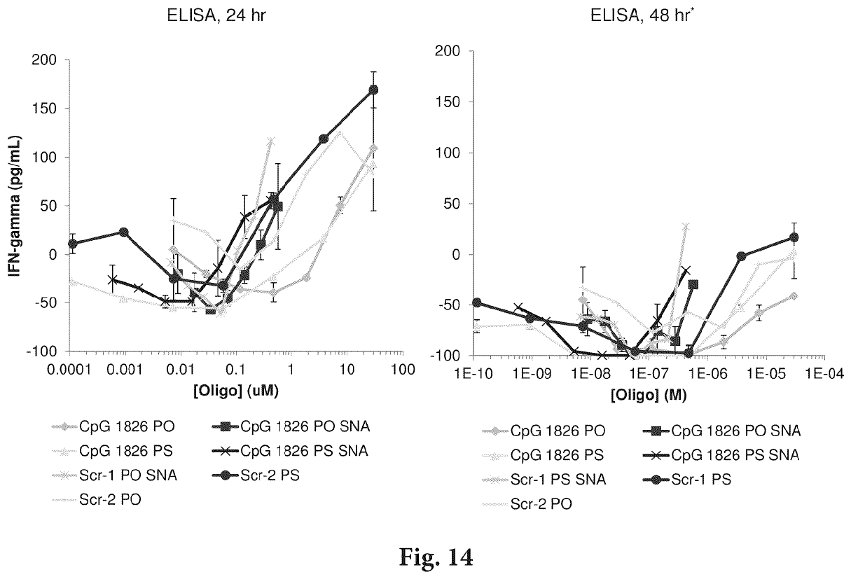

It was demonstrated that low levels of IFN-gamma secretion by macrophages (FIG. 14). RAW-Blue macrophages were plated at 65 k cells per well and allowed to adhere overnight. On the day of experiments, the indicated compounds were incubated with the cells overnight. The degree of cytokine secretion (either 24 hours—left panel or 48 hours—right panel after treatment) was determined by collecting the supernatant and measuring the concentration of the indicated cytokines by ELISA. The results show that IFN-gamma is not produced to an appreciable extent by RAW Blue macrophages stimulated with these compounds.

### Example 2 Immuno-Oncology and Immunotherapies

Immunotherapeutic SNAs (i.e., AST-008) provide a novel and versatile technology platform. Their multi-valent immunomodulator delivery optimizes responses, while profound tumor reduction in a lymphoma model has been observed. Additionally, they trigger a potent and balanced T cell response in vivo greater than that of free oligonucleotide or alum. SNAs can co-present therapeutic vaccine antigen and adjuvant on a single nanoparticle and have enhanced activity and faster kinetics than free immunostimulatory CpG oligodeoxynucleotides. Furthermore, SNAs have the potential to simultaneously target multiple immunostimulatory receptors (e.g. TLR 3, 4, 7/8, 9). They can be used, for example, in cancer immunotherapy and vaccines (prophylactic or therapeutic).

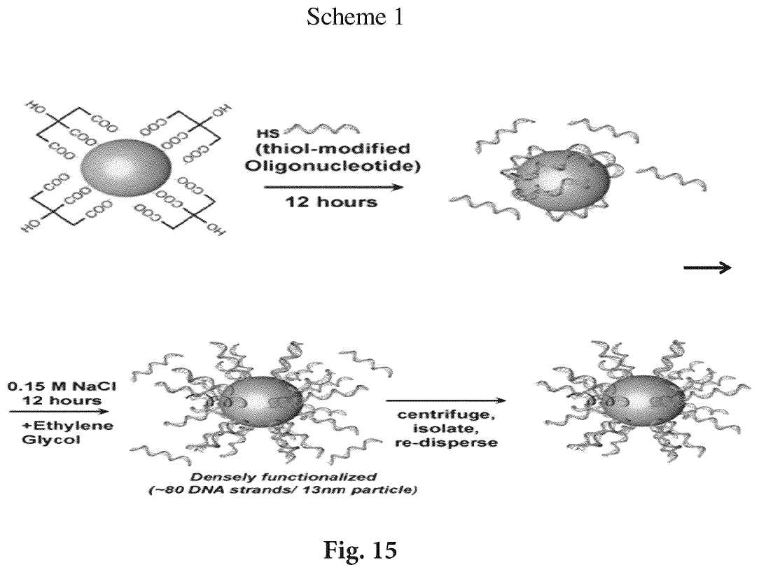

A schematic of an immunotherapeutic SNA (AST-008) is shown in FIG. 15. The SNAs can co-present a therapeutic vaccine antigen and adjuvant on a single nanoparticle, and may simultaneous target multiple immunostimulatory receptors (e.g. TLR 3, 4, 7/8, 9).

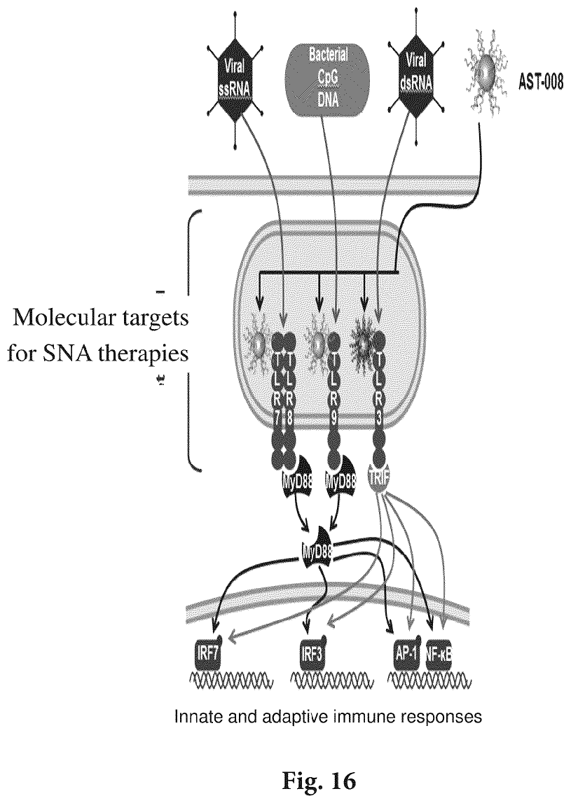

A schematic demonstrating how AST-008 can enter endosomes via triggered endocytosis is shown in FIG. 16. AST-008 once in the endosomes can be used for versatile immune system stimulation. Within the endosome, AST-008 stimulates immune system signaling via the TLR 9 receptor, a molecular target for SNA therapy, leading to both innate and adaptive immune responses. AST-008 may also target TLR 3, 4, 7/8, resulting in innate and adaptive immune responses.

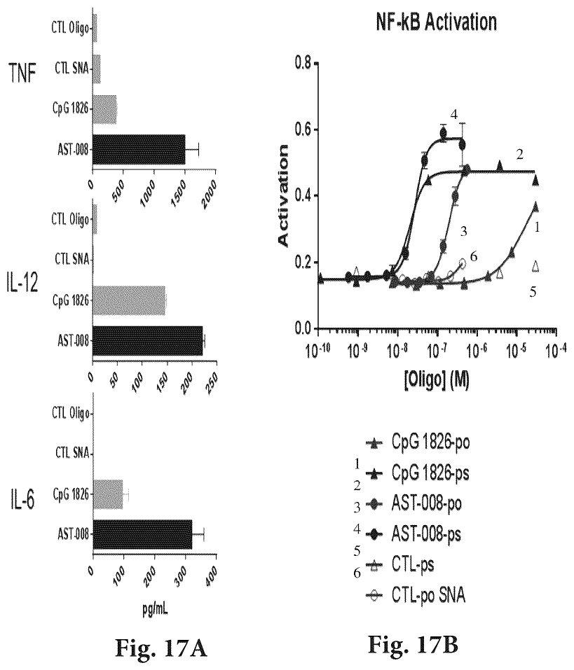

AST-008 induces higher pro-inflammatory responses than corresponding CpG oligodeoxynucleotides (oligo) in vitro. An assay demonstrating this finding was conducted. The data is shown as a set of graphs in FIGS. 17A and 17B. FIG. 17A shows the expression levels of TNF, IL-12, and IL-6 induced by CTL oligo, CTL SNA, CpG 1826, and AST-008. FIG. 17B presents the NF-κB activation stemming from the indicated agents.

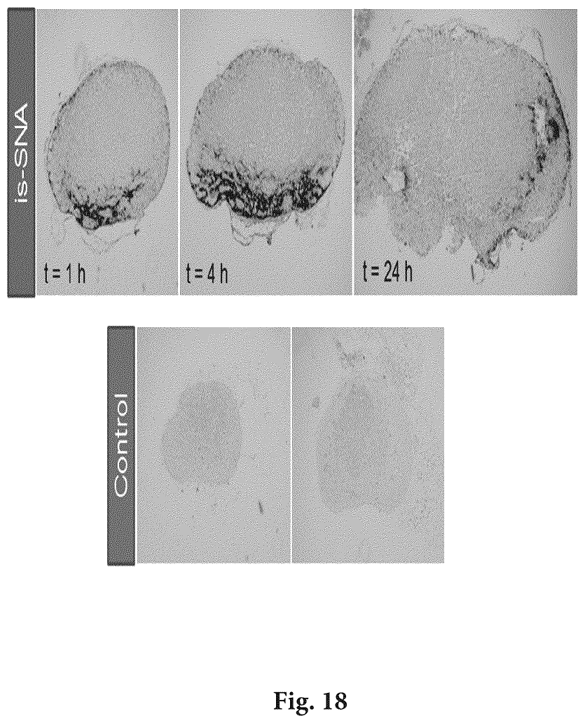

AST-008 also targets draining lymph nodes after administration of a single subcutaneous dose. AST-008 was silver-stained to enhance light scattering of the gold core, and then counterstained with eosin. 4× bright field magnification was used. The data is shown in FIG. 18.

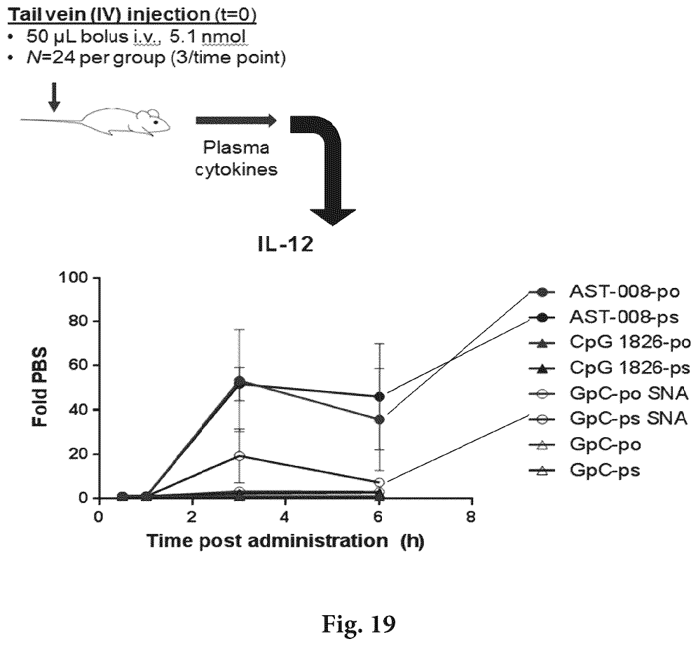

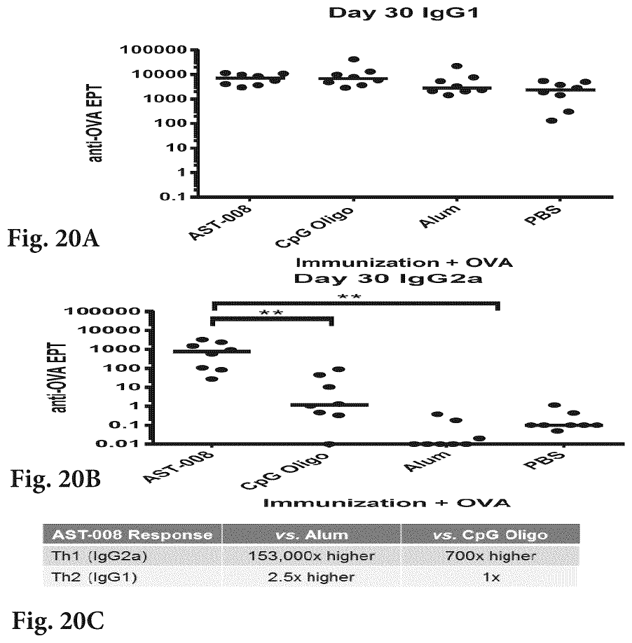

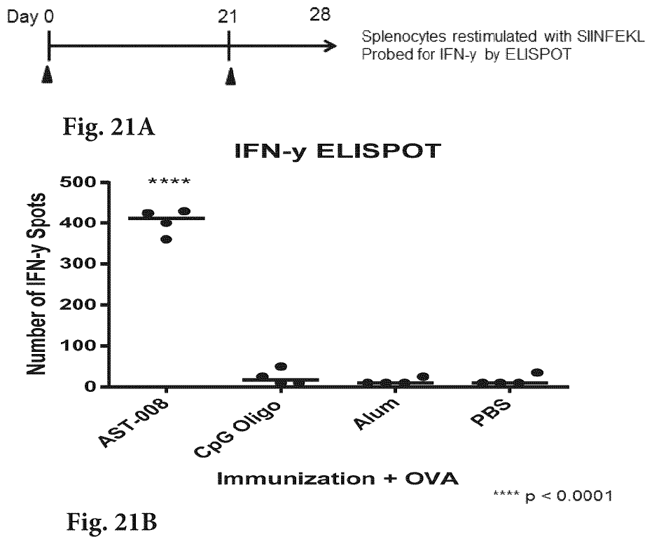

The structures of the invention are useful for stimulating a robust immune response in vivo. For instance, FIG. 19 is a graph illustrating the in vivo activity of AST-008. Mice were given a 50 μL bolus tail vein (intravenous) injection of 5.1 nmol solution (AST-008-po, AST-008-ps, CpG 1826-po, CpG 1826-ps, GpC-po SNA, GpC-ps SNA, GpC-po, or GpC-ps) and then analyzed for IL-12 expression 1, 3, and 6 hours after injection (24 mice per group, 3 per each time point). IL-12 levels are expressed as the fold over PBS. AST-008 architecture enhances the induction of IL-12 by approximately 20-fold over free oligodeoxynucleotides, and the effect was sustained for over six hours after the initial administration. FIGS. 20A-20C consist of a pair of graphs and a chart that demonstrate that AST-008 induces both a balanced Th1/Th2 response (FIG. 20A) and a higher IgG2a antibody (FIG. 20B) response than alum or CpG oligonucleotides. The results are tabulated in FIG. 20C. **p<0.01. FIGS. 21A-21B show that AST-008 induces cellular responses more effectively than alum or CpG oligonucleotides. FIG. 21A schematically represents the protocol: splenocytes were grown for 28 days, challenged on Day 0 and Day 21, and then restimulated with SIINFEKL and probed for INF-γ with ELISPOT on Day 28. FIG. 21B is a graph depicting the results. ****p<0.0001.

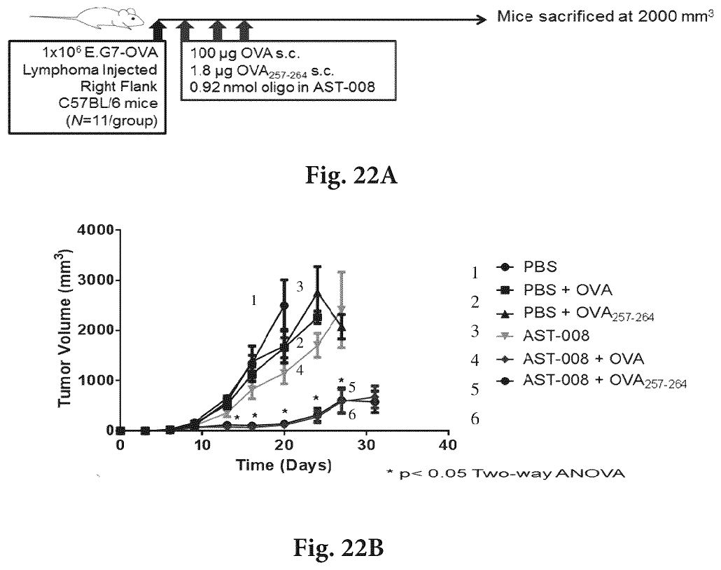

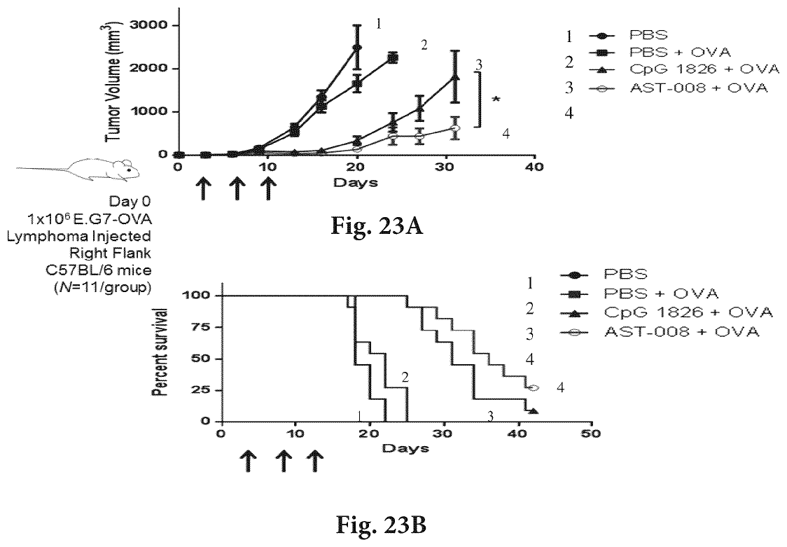

The structures have been shown to produce a dramatic anti-tumor response in vivo as well. FIGS. 22A-22B demonstrate that AST-008 induces a profound tumor-clearing immune response in an in vivo lymphoma model. FIG. 22A illustrates the protocol: the right flanks of C57BL/6 mice were injected with 1×10⁶ E.G7-OVA lymphoma (11 per group). The mice were then challenged three times with 100 μg OVA s.c., 1.8 μg OVA₂₅₇₋₂₆₄ s.c., and 0.92 nmol oligo in AST-008, and sacrificed at 2000 mm³. FIG. 13 is a graph of the results. *p<0.05 using Two-way ANOVA. FIGS. 23A-23B show that AST-008 exhibits superior anti-tumor activity and longer survival than CpG oligodeoxynucleotides. The graphs show the tumor volume (FIG. 23A) and percent survival (FIG. 23B) after C57BL/6 mice were injected with 1×10⁶ E.G7-OVA lymphoma in their right flanks (11 per group) and then were challenged three times with PBS, PBS and OVA, CpG 1826 and OVA, or AST-008 and OVA. *p<0.05.

TABLE 1
Key to symbols

| | | | SEQ ID |
|---|---|---|---|
| Name | Oligo Sequence (5′-3′) | Formulation | NO: |
| AST- | TCCATGACGTTCCTGATGCT/ | Conjugated to 13 | 36 |
| 007-po, | iSp18//iSp18//iSp18// | nm gold core via | |
| CpG | 3ThioMC3-D/ | thio-gold bond | |
| 1668 PO | | | |
| SNA | | | |
| AST- | TCCATGACGTTCCTGACGTT/ | Conjugated to 13 | 37 |
| 008-po, | iSp18//iSp18// | nm gold core via | |
| CpG | 3ThioMC3-D/ | thio-gold bond | |
| 1826 PO | | | |
| SNA | | | |
| AST- | TCGTCGTTTTGTCGTTTTGTCG | Conjugated to 13 | 38 |
| 009-po, | TT/iSp18//iSp18// | nm gold core via | |
| CpG | 3ThioMC3-D/ | thio-gold bond | |
| 7909 PO | | | |
| SNA | | | |
| AST- | tccatgacgttcctgatgct/ | Conjugated to 13 | 39 |
| 007-ps, | iSp18//iSp18//iSp18// | nm gold core via | |
| CpG | 3ThioMC3-D/ | thio-gold bond, | |
| 1668 PS | | backfilled with | |
| SNA | | tetraethylene | |
| | | glycol | |
| AST- | tccatgacgttcctgacgtt/ | Conjugated to 13 | 40 |
| 008-ps, | iSp18//iSp18//iSp18// | nm gold core via | |
| CpG | 3ThioMC3-D/ | thio-gold bond, | |
| 1826 PS | | backfilled with | |
| SNA | | tetraethylene | |
| | | glycol | |
| AST- | tcgtcgttttgtcgttttgtcg | Conjugated to 13 | 41 |
| 009-ps, | tt/iSp18//iSp18// | nm gold core via | |
| CpG | 3ThioMC3-D/ | thio-gold bond, | |
| 7909 PS | | backfilled with | |
| SNA | | tetraethylene | |
| | | glycol | |
| CpG | TCCATGACGTTCCTGACGTT | Free | 42 |
| 1826-po | | | |
| CpG | Tccatgacgttcctgacgtt | Free | 43 |
| 1826-ps | | | |
| CpG | TCCATGACGTTCCTGATGCT | Free | 44 |
| 1668-po | | | |
| CpG | Tccatgacgttcctgatgct | Free | 45 |
| 1668-ps | | | |
| CpG | TCGTCGTTTTGTCGTTTTGTCG | Free | 46 |
| 7909-po | TT | | |
| CpG | Tcgtcgttttgtcgttttgtcg | Free | 47 |
| 7909-ps | tt | | |
| rplV-po | GCTTTCTTGTTGGTGTAGGTC | Free | 48 |
| rplV-ps | Gctttcttgttggtgtaggtc | Free | 49 |
| Ctrl- | Sequence containing | Conjugated to 13 | |
| SNA-po, | no CpG motifs, all | nm gold core via | |
| rplV | phosphodiester | thio-gold bond | |
| SNA PO | linkages | | |
| Ctrl- | Sequence containing | Conjugated to 13 | |
| SNA-ps, | no CpG motifs, all | nm gold core via | |
| rplV | phosphorothioate | thio-gold bond | |
| SNA PS | linkages | | |

Lowercase indicates phosphorothioate linkages
Capital indicates phosphodiester linkages
Prefix “Ctrl” indicates oligo used with no CpG motifs present
/iSp18/ internal spacer-18 (hexaethylene glycol)
/3ThioMC3-D/ terminal sulfhydryl group

### REFERENCES

1. Koff W C et al. Science 340:1232910-1 (2013)
2. Cluff C W. Monophosphoryl Lipid A (MPL) as an Adjuvant for Anti-Cancer Vaccines: Clinical Results. Lipid A in Cancer Therapy, Jeannin J Ed. Landes Bioscience (2000)
3. Krieg A M. Proc Am Thorac Soc 4:289 (2007)
4. Schmidt C. Nat Biotechnol 25:825 (2007)
5. Ellis R D et al. PLOS One 10:e46094 (2012)
6. Garcon N et al. Expert Rev. Vaccines 6:723 (2007)
7. Rosi N L et al. Science 312:1027 (2006)
8. Lytton-Jean A K et al. JACS 127:12754 (2005)
9. Hurst S J et al. Anal. Chem. 78:8313 (2006)
10. Patel P C et al. PNAS 105:17222 (2008)
11. Seferos D S et al. Nano Lett 9:308 (2009)
12. Giljohann et al. Angew. Chem. Int. Ed. 49:3280 (2010)

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A nanoscale construct comprising:
a corona comprised of an exterior shell composed of nucleic acid molecules arranged in a geometrical position and forming a spherical shape around a nanoparticle core, wherein the nucleic acid molecules are CpG oligonucleotides, and wherein the nanoscale construct is about 1 nm to about 40 nm in mean diameter, and wherein the nanoparticle core at the center of the corona is not metallic.

2. The nanoscale construct of claim 1, wherein the surface density of the nucleic acid is at least 0.3 pmol/cm2.

3. The nanoscale construct of claim 1, wherein the CpG oligonucleotides comprise a spacer.

4. The nanoscale construct of claim 3, wherein the spacer is a tetraethylene glycol.

5. The nanoscale construct of claim 1, wherein the core at the center of the corona is hollow.

6. The nanoscale construct of claim 1, wherein the nanoscale construct is degradable.

7. The nanoscale construct of claim 1, wherein the CpG oligonucleotide has a modified backbone such as a phosphorothioate (PS) backbone.

8. The nanoscale construct of claim 1, further comprising an antigen.

9. A method for delivering a therapeutic agent to a cell comprising delivering the nanoscale construct of claim 1 to the cell.

10. A method of treating a subject, comprising
administering to the subject the nanoscale construct in an effective amount to stimulate an immune response, wherein the nanoscale construct comprises a corona having an exterior shell composed of nucleic acid molecules arranged in a geometrical position and forming a spherical shape around a nanoparticle core, wherein the nucleic acid molecules are CpG oligonucleotides, and wherein the nanoscale construct is about 1 nm to about 40 nm in mean diameter, and wherein the nanoparticle core at the center of the corona is not metallic.

11. The method of claim 10, wherein the subject has an infectious disease.

12. The method of claim 10, wherein the subject has cancer.

13. A method for activating a TLR comprising delivering to a cell a nanoscale construct, wherein the nanoscale construct comprises a corona having an exterior shell composed of nucleic acid molecules arranged in a geometrical position and forming a spherical shape around a nanoparticle core, wherein the nucleic acid molecules are CpG oligonucleotides, and wherein the nanoscale construct is about 1 nm to about 40 nm in mean diameter, and wherein the nanoparticle core at the center of the corona is not metallic.

14. The method of claim 13, wherein the cell is in vivo.

15. The method of claim 14, wherein the nanoscale construct is adminstered to a subject having cancer.
